(19) United States
(12) Patent Application Publication
(10) Pub. No.: US 2026/0144257 A1
(43) Pub. Date: May 28, 2026

(54) VITAMIN B3 BASED PRESERVATIVE COMPOSITIONS

(71) Applicant: SHARON PERSONAL CARE LTD., Ashdod (IL)

(72) Inventors: Ariel GLIKSBERG, Ashdod (IL); Paul SALAMA, Ashdod (IL); Noa ZIKLO, Tel-Aviv-Jaffa (IL)

(21) Appl. No.: 19/121,576

(22) PCT Filed: Oct. 19, 2023
(86) PCT No.: PCT/IL2023/051089

§ 371 Date: Apr. 16, 2025

Related U.S. Application Data
(60) Provisional application No. 63/417,372, filed on Oct. 19, 2022.

(51) Int. Cl. A01N 43/40; A01N 25/02; A01P 1/00

(52) U.S. Cl. CPC A01N 43/40; A01N 25/02; A01P 1/00

(57) ABSTRACT

Disclosed herein is a composition comprising niacinamide and a surfactant selected from cationic surfactant and a saponin in a ratio between 30:1 and 5:1 by weight, respectively. Formulations and articles comprising the compositions are disclosed. Further methods of inhibiting or reducing the formation of load of a microorganism in and/or on an article and methods of preserving a personal care product are also disclosed.

### CROSS REFERENCE TO RELATED APPLICATIONS

This application claims the benefit of priority of U.S. Provisional Patent Application No. 63/417,372, filed on Oct. 19, 2022, the content of which is incorporated herein by reference in its entirety.

### FIELD OF THE INVENTION

The present invention is in the field of preservation of antimicrobial compositions and/or personal care formulations.

### BACKGROUND OF THE INVENTION

Antimicrobial compositions are used, for example, in the health care industry, food service industry, cosmetic industry, and in the private sector by individual consumers.

Preservation of water-based compositions (e.g. liquid, solid, or semi-solid compositions) and/or personal care products from microbial contamination has become a difficult task since the available approved antimicrobials are very few and those which have good antimicrobial activity are quite toxic. For example, consumers seek for products for topical applications being free from toxic antimicrobials that can be used as preservatives.

It is important, however, that antimicrobial compositions provide a substantial and broad-spectrum reduction in microorganism populations quickly and without problems associated with toxicity and allergic reaction.

To this end, there is a great need for low cost and effective preservative formulations composed of GRAS constituents and substantially free of phenoxyethanol, benzyl alcohol, or parabens that can prevent or reduce microbial infestation of cosmetic formulations, pharmaceutical formulations, food formulations, and/or or of personal care products.

### SUMMARY OF THE INVENTION

In one aspect of the invention, there is provided an antimicrobial composition comprising niacinamide, a salt thereof or both; and a surfactant selected from a cationic surfactant and a saponin; wherein a ratio between (i) niacinamide, a salt thereof or both and (ii) the surfactant is between 30:1 and 5:1 by weight; and a w/w concentration of niacinamide, the salt thereof or both within the antimicrobial composition is at least 2%.

In one embodiment, the cationic surfactant comprises Laurdimoniumhydroxypropyl Decylglucoside (Polyquaternium 78), Ethyl Lauroyl Arginate (ELA), and Cola lipid C, including any salt, or any combination thereof.

In one embodiment, the antimicrobial composition further comprising an agent selected from Caprylhydroxamic acid, a zinc compound, including any salt or any combination thereof.

In one embodiment, the agent is present within the antimicrobial composition at an amount between 0.1:1 and 1:1 by weight, relative to the surfactant.

In one embodiment, the zinc compound comprises a zinc (II) cation including any salt, any complex thereof, or both.

In one embodiment, the zinc compound comprises zinc halide, zinc acetate, zinc pyrithione, zinc gluconate, zinc lactate, zinc glycinate or any combination thereof.

In one embodiment, the antimicrobial composition further comprises a pharmaceutically or a cosmeceutically acceptable carrier.

In one embodiment, the antimicrobial composition comprises an antimicrobial effective amount of niacinamide, of the surfactant, or both.

In one embodiment, the antimicrobial effective amount of niacinamide within the antimicrobial composition is between about 2.5 and about 6% w/w.

In one embodiment, the antimicrobial effective amount of the surfactant within the antimicrobial composition is between about 0.05 and about and 1% w/w.

In one embodiment, the antimicrobial composition is an aqueous composition.

In one embodiment, the antimicrobial composition is characterized by a sterilizing or preservation activity within an article or on a surface upon contacting an antimicrobial effective amount of the antimicrobial composition with the article or with the surface.

In one embodiment, the microorganism comprises an eukaryotic organism, a fungi, a pathogen, or a bacterium, including any combination thereof.

In another aspect, there is provided an article comprising an antimicrobial effective amount of the composition of the invention, and a pharmaceutically or a cosmeceutically acceptable carrier; wherein: the antimicrobial effective amount comprises a concentration of niacinamide within the article of at least about 2% w/w; and the article is characterized by a substantially reduced pathogen load, compared to a similar article devoid of the antimicrobial composition.

In one embodiment, the substantially reduced pathogen load is maintained for a period ranging between 1 week and 2 years.

In one embodiment, the article is sterile.

In one embodiment, the antimicrobial effective amount comprises (i) a w/w concentration of niacinamide within the article of at least about 2.5% w/w; and (ii) a w/w concentration of the surfactant within the article of at least about 0.05% w/w.

In one embodiment, the antimicrobial effective amount comprises a w/w concentration of niacinamide within the article between about 2.5 and about 6%.

In one embodiment, the antimicrobial effective amount further comprises a w/w concentration of the agent within the article of at least about 0.01% w/w.

In one embodiment, the article is a cosmeceutical product, cosmetic product, a pharmaceutical product, a personal care product, or any combination thereof.

In one embodiment, the personal care product is selected from a fabric, a bandage, a wipe, a swab, a suppository, a dressing, a solution, a mousse, a pad, and a patch, or any combination thereof.

In one embodiment, the cosmetical or cosmeceutical product is in a form of a formulation selected from the group consisting of: a liquid, a solution, a paste, a cream, a lotion, a foam, a gel, an emulsion, an ointment, and a soap.

In one embodiment, the article is characterized by a water content of at least 10%.

In another aspect, there is provided a kit comprising the antimicrobial composition of the invention, and further comprising instructions for dilution of the antimicrobial composition with an appropriate carrier, to obtain a liquid composition comprising an antimicrobial effective amount of the antimicrobial composition.

In another aspect, there is provided a method of inhibiting or reducing the formation or load of a microorganism in and/or on an article, the method comprising contacting the article with an antimicrobial effective amount of the antimicrobial composition of the invention, or with the kit of the invention.

In another aspect, there is provided a method of preserving a personal care product, comprising adding to the personal care product the antimicrobial effective amount of the antimicrobial composition of the invention, or of the kit of the invention.

In another aspect, there is provided an antimicrobial composition comprising niacinamide, a salt thereof or both; and a surfactant selected from a cationic surfactant and a saponin; wherein:
a ratio between (i) niacinamide, a salt thereof or both and (ii) the surfactant is between 30:1 and 5:1 by weight; and
the antimicrobial composition is characterized by an antimicrobial synergistically effective amount of at least 2% w/w.

In one embodiment, the cationic surfactant comprises Lauryldimoniumhydroxypropyl Decylglucoside (Polyquaternium 78), Ethyl Lauroyl Arginate (ELA), and Cola lipid C, including any salt, or any combination thereof.

In one embodiment, the antimicrobial composition further comprises an agent selected from Caprylhydroxamic acid, a zinc compound, including any salt or any combination thereof.

In one embodiment, the agent is present within the antimicrobial composition at an amount between 0.1:1 and 1:1 by weight, relative to the cationic surfactant.

In one embodiment, the zinc compound comprises a zinc (II) cation including any pharmaceutically acceptable salt, any complex thereof, or both.

In one embodiment, the zinc compound comprises zinc halide, zinc acetate, zinc pyrithione, zinc gluconate, zinc lactate, zinc glycinate or any combination thereof.

In one embodiment, the antimicrobial composition further comprises a pharmaceutically or a cosmeceutically acceptable carrier.

In one embodiment, the antimicrobial synergistically effective amount of the antimicrobial composition is between about 2.5 and about 6% w/w.

In one embodiment, the antimicrobial composition is an aqueous composition; wherein a concentration of niacinamide within the aqueous composition is between 10 and 60% w/w; and wherein a concentration of the cationic surfactant or of the saponin within the aqueous composition is independently between 0.5 and 6% w/w; and wherein the antimicrobial synergistically effective amount of the antimicrobial composition is between about 2.5 and 4% w/w.

In another aspect, there is provided an antimicrobial composition comprising niacinamide, a salt thereof or both; and a glycol; wherein:

a ratio between niacinamide, a salt thereof or both and the glycol is between 30:1 and 5: 1w/w by weight; and
wherein the antimicrobial composition is characterized by an antimicrobial synergistically effective amount of between about 2 and about 5% w/w.

In one embodiment, the antimicrobial composition is a preservative composition characterized by a sterilizing or preservation activity within an article when present within the article at the antimicrobial synergistically effective amount.

In one embodiment, the glycol comprises (i) C4-C15diol, (ii) R—C(O)—O—X, or both, wherein R is an allyl between 3 and 20 carbon atoms long; X is a C2-C10 alkyl substituted by 2 hydroxy groups, or both (i) and (ii).

In one embodiment, the (ii) is a C5-C18 fatty acid monoglyceride.

In one embodiment, the C5-C18 fatty acid monoglyceride is glyceryl caprylate.

In one embodiment, the antimicrobial synergistically effective amount is between about 2.5 and 4% w/w; wherein the glycol comprises glyceryl caprylate and the C4-C15diol; wherein a w/w ratio between niacinamide and glyceryl caprylate is between about 8:1 and about 20:1; and wherein a w/w ratio between glyceryl caprylate and the C4-C15diol is between 1:1 and 5:1.

In one embodiment, the diol comprises the C4-C15diol; and wherein the C4-C15diol comprises a C4-C15 alkyl or heteroalkyl substituted by two hydroxy groups.

In one embodiment, the antimicrobial composition further comprising Caprylhydroxamic acid.

In one embodiment, wherein a w/w ratio between the C4-C15diol and Caprylhydroxamic acid is between 1:1 and 5:1.

In one embodiment, the C4-C15diol comprises any one of: caprylyl glycol, ethylhexyl glycerin and decanediol, or any combination thereof.

In one embodiment, the antimicrobial composition is an aqueous composition; wherein a concentration of niacinamide and of the glycol with said aqueous composition is between 10 and 60% w/w and between 0.5 and 5% w/w, respectively.

In one embodiment, the aqueous composition further comprises a between 0.5 and 5% w/w of a surfactant.

In another aspect, there is provided an antimicrobial composition comprising niacinamide, a salt thereof or both; and a polylysine; wherein:
a ratio between niacinamide, a salt thereof or both and the polylysine is between 100:1 and 20:1 by weight; and
wherein the antimicrobial composition is characterized by an antimicrobial synergistically effective amount of between about 2 and about 5% w/w.

In one embodiment, the antimicrobial composition further comprising hexylresorcinol.

In one embodiment, a w/w ratio between the polylysine and hexylresorcinol is between 2:1 and 1:2.

In one embodiment, the polylysine is characterized by an average MW between 2000 and 10000 Da.

In one embodiment, the antimicrobial composition is an aqueous composition; wherein a concentration of niacinamide with the aqueous composition is between 10 and 50% w/w; and wherein a concentration of the polylysine and of hexylresorcinol with the aqueous composition is between 0.2 and 1% w/w, respectively.

In one embodiment, the antimicrobial composition is characterized by a sterilizing or preservation activity within an article when applied to the article at the antimicrobial synergistically effective amount; and wherein the article is selected from a liquid formulation and a semi-solid formulation.

In one embodiment, the antimicrobial synergistically effective amount is sufficient for at least 20% increased reduction of a pathogen load within the article, as compared to sole addition of the same amount of niacinamide.

In one embodiment, the antimicrobial synergistically effective amount is a preservative effectiveness according to EP and/or USP acceptance criteria, as determined by a challenge test.

In one embodiment, the pathogen comprises an eukaryotic organism, a fungi, a mold, or a bacterium, including any combination thereof.

In another aspect, there is provided an article comprising the synergistically effective amount of the antimicrobial composition of the invention, and a pharmaceutically or a cosmeceutically acceptable carrier.

In one embodiment, the article is characterized by a substantially reduced load of a pathogen, compared to a control; and wherein the substantially reduced load comprises at least 10 fold pathogen load reduction 48 h after infestation by the pathogen, compared to control; and wherein the control is a similar article devoid of the antimicrobial composition.

In one embodiment, the synergistically effective amount comprises a w/w concentration of niacinamide within the article between about 2.5% and about 3.5% w/w.

In one embodiment, the article is in a form of a liquid formulation or a semi-solid formulation, optionally wherein the article is a cosmetic product, a pharmaceutical product, a personal care product, or any combination thereof.

In one embodiment, the article is characterized by a water content of at least 10%; and wherein the synergistically effective amount is sufficient for obtaining a dry weight concentration of the antimicrobial composition within the article of between about 2 and about 5% w/w.

In another aspect, there is provided a method for reducing pathogen load in and/or on an article or for preserving the article, the method comprising contacting the article with a synergistically effective amount of the antimicrobial composition of the invention.

In one embodiment, the article is in a form of a moist textile substrate, liquid formulation or a semi-solid formulation, optionally wherein the article is a cosmetic product, a pharmaceutical product, a personal care product, or any combination thereof; and is characterized by a water content of at least 10%.

In one embodiment, reducing comprises at least 10 times CFU reduction of one or more pathogens within the article 48h after the contacting, compared to a similar article devoid of the antimicrobial composition.

In one embodiment, preserving is according to EP and/or USP acceptance criteria, as determined by a challenge test.

In one embodiment, the synergistically effective amount is sufficient for obtaining a dry weight concentration of the antimicrobial composition within the article of between about 2 and about 5% w/w.

In one embodiment, the synergistically effective amount is sufficient for obtaining a dry weight concentration of the antimicrobial composition within the article of between about 2.5 and about 4% w/w.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a table presenting a preservative activity of an exemplary composition of the invention comprising about 2.8% w/w of niacinamide and about 0.2% w/w Monomer-78, when tested in a basic cream non-ionic formulation.

FIG. 2 is a table presenting a preservative activity of an exemplary composition of the invention comprising about 2.8% w/w of niacinamide and about 0.2% w/w Ethyl Lauroyl Arginate (ELA), when tested in a basic cream non-ionic formulation.

FIG. 3 is a table presenting a preservative activity of an exemplary composition of the invention comprising about 2.8% w/w of niacinamide and about 0.2% w/w Monomer-78, when tested in an anionic shampoo formulation.

FIG. 4 is a table presenting a preservative activity of an exemplary composition of the invention comprising about 2.8% w/w of niacinamide and about 0.2% w/w Monomer-78, when tested in a cationic mask formulation.

FIG. 5 is a table presenting a preservative activity of an exemplary composition of the invention comprising about 2.7% w/w of niacinamide and about 0.3% w/w Saponin, when tested in a basic cream non-ionic formulation.

FIG. 6A is a table presenting a preservative activity of an exemplary composition of the invention comprising about 2.8% w/w of niacinamide, about 0.1% w/w Cola lipid C, and about 0.05% w/w capryl hydroxamic acid, when tested in a basic cream non-ionic formulation.

FIG. 6B is a table presenting a preservative activity of a control composition comprising about 0.1% w/w of Cola lipid C and about 0.05% w/w capryl hydroxamic acid, when tested in a basic cream non-ionic formulation.

FIG. 7 is a table presenting a preservative activity of an exemplary composition of the invention comprising about 2.8% w/w of niacinamide, about 0.1% w/w Cola lipid C, and about 0.05% w/w zinc chloride, when tested in a basic cream non-ionic formulation.

FIG. 8 is a table presenting a preservative activity of an exemplary composition of the invention comprising about 2.8% w/w of niacinamide about 0.1% w/w Cola lipid C, and about 0.1% w/w zinc acetate, when tested in a basic cream non-ionic formulation.

FIG. 9 is a table presenting a preservative activity of an exemplary composition of the invention comprising about 2.8% w/w of niacinamide and about 0.05% ELA, when tested in a basic cream non-ionic formulation.

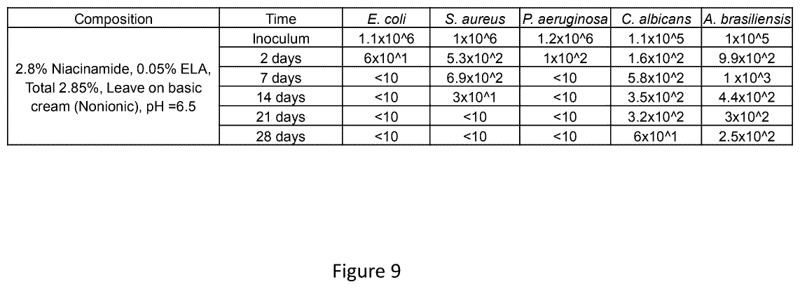

FIG. 10 is a table presenting a preservative activity of an exemplary composition of the invention comprising about 2.8% w/w of niacinamide, 0.15% glyceryl caprylate and about 0.05% decanediol, when tested in an anionic shampoo formulation.

FIG. 11 is a table presenting a preservative activity of an exemplary composition of the invention comprising about 2.8% w/w of niacinamide, 0.15% w/w Caprylyl glycol and 0.05% w/w Caprylhydroxamic acid, when tested in a basic cream non-ionic formulation.

FIG. 12 is a table presenting a preservative activity of an exemplary composition of the invention comprising about 2.9% w/w of niacinamide and 0.035% w/w polylysine, when tested in a basic cream non-ionic formulation.

### DETAILED DESCRIPTION OF THE INVENTION

The present invention is directed to a composition comprising a predetermined ratio between (i) niacinamide, a salt thereof or both; and (ii) a surfactant selected from a cationic surfactant, a saponin, a glycol and polylysine; wherein the composition is characterized by a sanitizing, antimicrobial, and/or preserving activity upon contacting an antimicrobially effective amount of the composition with a formulation selected from an aqueous formulation, a gel, a cream, an ointment, and a foam, or with a wet substrate.

The present invention is also directed to formulations comprising an antimicrobially effective amount of the composition described herein. In some embodiments, the formulation is selected from a cosmetic formulation, cosmeceutical formulation, pharmaceutical formulation, food formulation, and/or personal care formulation.

The present invention is also directed to articles comprising the composition or a formulation described herein. In some embodiments, the article is selected from a cosmetic article, a pharmaceutical article, a food article, or a personal care article. In some embodiments, the article is a solid, a semi-solid, a semi-liquid, or a liquid article. In some embodiments, the article is an aqueous formulation, a gel, a foam or a cream.

The present invention is based, in part, on the finding that compositions (e.g., formulations) according to the present invention, comprising niacinamide and/or a salt thereof and a surfactant disclosed herein, being at certain predetermined ratios (e.g. synergistically effective amount), exhibit a desired stability as well as improved antimicrobial activities, compared to similar formulations composed of niacinamide as the sole active ingredient.

Furthermore, the present invention is based, in part, on the finding that a liquid (e.g. an aqueous formulation) or a semi-liquid/semi-solid formulation comprising about 3% w/w of the antimicrobial composition of the invention, is characterized by a preservative activity according to USP/EP acceptance criteria, as determined by a challenge test (ISO 11930).

The term “antimicrobial” refers to a composition or compound capable of preventing the growth, inhibiting the growth, and/or controlling the growth of microorganisms; antimicrobial compounds include bactericides, bacteriostats, fungicides, fungistats, algaecides and algistats. The term “antimicrobial” encompasses preservatives and sterilization agents.

Herein throughout, by “composition” it is further meant to refer to a formulation, or a solid composition.

In some embodiments, the present invention provides non-toxic antimicrobial formulations (also referred to as “blends”) or concentrates. In some embodiments, the antimicrobial composition is a dilutable concentrate. In some embodiments, the antimicrobial composition is a dilutable by at least 100 fold, at least 1000 fold, at least 10000 fold, at least 100000 fold, including any range between.

As used herein, the terms “formulation”, or “blend”, which are used hereinthroughout interchangeably, refer to a vehicle composition in the form of a solid, or a liquid composition, such as a solution, an emulsion, a dispersion, a suspension, etc., that optionally further comprises a surface active agent. The formulation can optionally further comprise a carrier, and optionally additional active agents and/or additives.

In some embodiments, the term “by weight” means by weight of the total composition.

### The Composition

In one aspect of the invention, there is provided an antimicrobial composition comprising (i) niacinamide, and/or an antimicrobial derivative thereof, and/or a salt thereof; and (ii) a surfactant; wherein a weight ratio (i.e. the predetermined ratio) between (i) and (ii) is between 30:1 and 5:1, between 30:1 and 10:1, between 30:1 and 15:1, between 30:1 and 20:1, between 20:1 and 5:1, 20:1 and 8:1, 15:1 and 5:1, 20:1 and 10:1, 15:1 and 10:1, 10:1 and 5:1, 9:1 and 5:1, 15:1 and 5:1, between about 98:2 and about 80:20, between about 95:5 and about 80:20, between about 95:5 and about 87:13, between about 95:5 and about 86:14, between about 95:5 and about 85:15, between about 93:17 and about 85:15, between about 90:10 and about 85:15, 7:1 and 5:1, 6:1 and 5:1, 10:1 and 6:1, 9:1 and 6:1, 8:1 and 6:1, or between 7:1 and 5:1 by weight, respectively, including any range therebetween. Each possibility represents a separate embodiment of the invention.

In some embodiments, the antimicrobial composition comprises a single surfactant specie, or a plurality of surfactants (e.g. at least two chemically distinct surfactants), including any salt(s) thereof. In some embodiments, the surfactant is selected from a cationic surfactant and a saponin. In some embodiments, the surfactant is a glycol.

In some embodiments, the antimicrobial composition comprises (i) niacinamide, and/or an antimicrobial derivative thereof, and/or a salt thereof; and (ii) a surfactant; wherein a weight ratio between (i) and (ii) is between 100:1 and 20:1, between 80:1 and 20:1, between 60:1 and 20:1, between 80:1 and 40:1, between 80:1 and 50:1, between 80:1 and 30:1, between 100:1 and 50:1 between about 98:2 and about 80:20, between about 95:5 and about 80:20, between about 95:5 and about 87:13, between about 95:5 and about 86:14, between about 95:5 and about 85:15, between about 93:17 and about 85:15, between about 90:10 and about 85:15 by weight, respectively, including any range therebetween; and wherein the surfactant is polylysine, including any salt or any copolymer thereof. In some embodiments, the polylysine is characterized by an average molecular weight between 2000 and 10000 Da, between 2000 and 8000 Da, between 2000 and 6000 Da, between 2000 and 4000 Da, between 2000 and 3500 Da, including any range between. In some embodiments, the antimicrobial composition comprises (i) niacinamide, and/or an antimicrobial derivative thereof, and/or a salt thereof; (ii) polylysine and (iii) hexylresorcinol; wherein a w/w ratio between (ii) and (iii) is between 1:3 and 3:1, between 3:1 and 1:1, between 3:1 and 2:1, between 2:1 and 1:1, between 1:1 and 3:1, between 1:1 and 2:1, or about 1:1. In some embodiments, the antimicrobial composition is essentially composed of naturally derived constituents. In some embodiments, the entire constituents of the antimicrobial composition are GRAS. In some embodiments, the antimicrobial composition is essentially composed of isolated or purified natural constituents. In some embodiments, the antimicrobial composition is substantially devoid of a synthetic antimicrobial agent, and/or synthetic preservatives. In some embodiments, the antimicrobial composition is substantially devoid of a synthetic constituent or a synthetic active agent. In some embodiments, the antimicrobial composition is substantially devoid of an non-natural constituent or a non-natural active agent.

In some embodiments, the term “derivative” refers to a stereoisomer (e.g. enantiomer, and/or diastereomer) and/or a structural isomer (e.g. an alkylated, an animated, a hydroxylated, a carboxylated derivative), an ester, a tautomer, and/or any prodrug or precursor of the above disclosed compounds. In some embodiments, the term “derivative” refers to a structural derivative having a similar (or an enhanced) antimicrobial activity.

In some embodiments, the cationic surfactant comprises a quaternary ammonium salt-based surfactant. In some embodiments, the cationic surfactant comprises a guanidine-based surfactant. In some embodiments, the cationic surfactant comprises a primary/secondary/tertiary- and/or quaternary amine-based surfactant; a guanidine-based cationic surfactant, or both.

In some embodiments, the antimicrobial synergistically effective amount of the antimicrobial composition of the invention is at least 2%, at least 2.5%, at least 2.6, at least 2.8%, or between about 2 and about 5%, between about 3 and about 5%, between about 3 and about 4%, between 2.5 and 3%, between 2.5 and 3.5%, between 2.5 and 4% w/w, including any range between.

The term “antimicrobial synergistically effective amount” encompasses an amount of the antimicrobial composition sufficient for inducing an enhanced antimicrobial activity (e.g. a preservative activity, or reduction of pathogen load) within a formulation selected from a liquid or a semi-liquid formulation, as compared to a control. The term “synergism”, or any grammatical derivative thereof, is defined as the simultaneous action of two or more compounds in which the total biological activity (e.g. preservative effectiveness, and/or antimicrobial activity) of the combination is greater than the sum of the individual components.

The term “enhanced” encompasses at least 20%, at least 50%, at least 100%, at least 10 fold, at least 100 fold, at least 1000 fold, at least 10000 fold enhanced antimicrobial activity, as compared to the control, including any range between.

The term “control” refers to the same formulation containing the same amount of niacinamide as the sole preservative, i.e. without the addition of the surfactant. The term “antimicrobial synergistically effective amount” further encompasses antimicrobial activity sufficient for a preservative effectiveness according to EP and/or USP acceptance criteria, as determined by a challenge test (e.g. according to ISO11930, or an alternative thereof). The antimicrobial composition of the invention is characterized by a broad spectrum preservative activity against bacteria, fungi and mold.

In some embodiments, the preservative activity of the antimicrobial composition refers to the ability thereof to reduce a load of at least one pathogen (e.g. bacterium species) in the liquid or the semi-liquid formulation by at least 10, at least 20, at least 50, at least 100 fold, including any range between, two days or 7 days post application of the antimicrobial synergistically effective amount of the antimicrobial composition. In some embodiments, the preservative activity of the antimicrobial composition refers to the ability thereof to (i) reduce a load of at least one bacterial pathogen (i.e. one or more bacterium species) in the liquid or the semi-liquid formulation by at least 10, at least 20, at least 50, at least 100 fold, including any range between, two days post application of the antimicrobial synergistically effective amount of the antimicrobial composition, and (ii) to maintain or reduce the initial load of at least one non-bacterial pathogen (i.e. one or more fungus specie, such as yeast, mold or both) in the liquid or the semi-liquid formulation up to 28 days post application of the antimicrobial synergistically effective amount of the antimicrobial composition.

In some embodiments, the preservative activity of the antimicrobial composition refers to the ability thereof to reduce (i) a load of at least one bacterial pathogen (i.e. one or more bacterium species) in the liquid or the semi-liquid formulation by at least 10, at least 20, at least 50, at least 100 fold, including any range between, two days post application of the antimicrobial synergistically effective amount of the antimicrobial composition, and (ii) a load of at least one non-bacterial pathogen (i.e. one or more fungus specie, such as yeast, mold or both) in the liquid or the semi-liquid formulation by at least 10, at least 20, at least 50, at least 100 fold, including any range between, 14 days post application of the antimicrobial synergistically effective amount of the antimicrobial composition.

In some embodiments, at least one pathogen comprises one or more human pathogen. In some embodiments, at least one pathogen is selected from bacteria, fungi and mold, or any combination thereof.

In some embodiments, the liquid or the semi-liquid formulation in conjunction with the preservative effectiveness, and/or antimicrobial activity refers to a formulation prone of microbial infestation (e.g. having a water content of at least 10%, at least 50%, or between 10 and 99%, between 20 and 90%, between 20 and 80%, between 30 and 99%, including any range between).

In some embodiments, the quaternary ammonium comprises an alkyl polyglucoside-based quaternary ammonium. In some embodiments, the alkyl polyglucoside-based quaternary ammonium is represented by Formula 1:

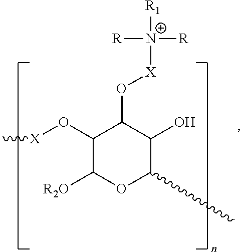

wherein X is an C1-C10 alkyl, or a C1-C10 heteroalkyl, optionally substituted by a hydroxyl group; each R, R1 and R2 is independently a C1-C25 alkyl; n is between 2 and 10. In some embodiments, X is 2-hydroxypropyl, and each R is methyl. In some embodiments, R1 and R2 is independently a C8-C22 alkyl. In some embodiments, X is 2-hydroxypropyl, and each R is methyl, R1 is lauryl, and R2 is a C8-C22 alkyl, and n is 4-6.

In some embodiments, the alkyl polyglucoside-based quaternary ammonium is Lauryldimoniumhydroxypropyl Decylglucoside (Polyquaternium 78) In some embodiments, the quaternary ammonium comprises a phosphate-based quaternary ammonium represented by Formula 2:

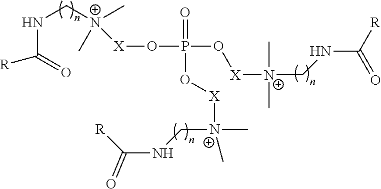

wherein X is an C1-C10 alkyl, or a C1-C10 heteroalkyl, optionally substituted by a hydroxyl group; n is 1-5; each R is independently a C5-C25 alkyl. In some embodiments, each R is independently a C5-C20 alkyl. In some embodiments, X is 2-hydroxypropyl, n is 2. In some embodiments, each R is a coco alkyl. In some embodiments, the phosphate-based quaternary ammonium is Cocamidopropyl PG-Dimonium Phosphate (Cola Lipid C).

In some embodiments, the guanidine-based cationic surfactant is represented by Formula 3:

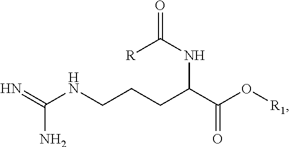

wherein R1 is a C1-C10 alkyl, and R is a C8-C20 alkyl. In some embodiments, R1 is a C1-C3 alkyl, and R is a C8-C15 alkyl. In some embodiments, R1 is a C1-C3 alkyl, and R is C11 alkyl. In some embodiments, the guanidine-based cationic surfactant is Ethyl Lauroyl Arginate (ELA).

In some embodiments, the quaternary ammonium is selected from, without being limited thereto, benzyldimethyldodecylammonium chloride, didecyldimethylammonium chloride, dodecyltrimethylammonium chloride, hexadecyltrimethylammonium chloride, Polyquaternium-2, Polyquaternium-80, Polyquaternium-11, Polyquaternium-52, Polyquaternium-17, Cocamidopropyl PG-Dimonium Phosphate (Cola Lipid C), Lauryldimoniumhydroxypropyl decylglucoside (PolySugaQuat L-1010), Berberine, Berberrubine, Berberis vulgaris root extract, Isopropylbenzyl butylnorberberine iodide, Palmatine, Jatrorrhizine, Coptisine, Ungeremine, Epiberberine, Pseudoberberine, Stepharanine, Sinapine, and any combination thereof.

In some embodiments, the quaternary ammonium is selected from Polyquaternium 78 and/or Cola lipid C. In some embodiments, the cationic surfactant comprises Lauryldimoniumhydroxypropyl Decylglucoside (Polyquaternium 78, or PolySugaQuat L-1010), Ethyl Lauroyl Arginate (ELA), and Cola lipid C.

In some embodiments, the diol is a C4-C15diol. In some embodiments, the C4-C15diol is or comprises a C4-C15 alkyl or heteroalkyl substituted by two hydroxy groups. The term “heteroalkyl” encompasses an alkyl comprising one or more heteroatom(s) within the alkyl chain. Alkyl may be a linear or a branched alkyl. In some embodiments, the C4-C15diol is or comprises a C4-C15 alkyl or heteroalkyl is a C8-C12, C8-C11, C5-C12, C6-C12, C6-C10 alkyl/heteroalkyl substituted by 2 hydroxy groups. In some embodiments, the C4-C15diol is 1,2 diol.

In some embodiments, the C4-C15diol is or comprises any one of: caprylyl glycol, ethylhexyl glycerin and decanediol (e.g. 1,2 decanediol), or any combination thereof.

In some embodiments, the diol is represented by Formula 4: R—C(O)—O—X, wherein R is a C3-20 alkyl and X is a C2-C10 alkyl substituted by 2 hydroxy. In some embodiments, R is C5-20 alkyl, C5-C10 alkyl, C6-C9 alkyl or C7 alkyl. In some embodiments, X is a dihydroxypropyl. In some embodiments, the diol of Formula 4 is a C5-C18, C5-C10, C6-C12, C6-C10, or C8 fatty acid monoglyceride. In some embodiments, the diol of Formula 4 is glyceryl caprylate.

In some embodiments, the diol is selected from decanediol, caprylyl glycol, ethylhexyl glycerin and glyceryl caprylate.

In some embodiments, the diol is a combination of a diol of Formula 4 and the C4-C15diol. In some embodiments, the diol is a combination of glyceryl caprylate and the C4-C15diol selected from decanediol, caprylyl glycol and ethylhexyl glycerin. In some embodiments, a w/w ratio between glyceryl caprylate and the C4-C15diol is between 1:1 and 5:1, between 2:1 and 5:1, between 2:1 and 4:1, between 2.5:1 and 3.5:1, or about 3:1, including any range between.

In some embodiments, the antimicrobial composition comprises niacinamide, glyceryl caprylate and the C4-C15diol; and a w/w ratio between glyceryl caprylate and the C4-C15diol is between 2:1 and 4:1; wherein a w/w ratio between niacinamide and glyceryl caprylate is between about 8:1 and about 20:1, between about 8:1 and about 15:1, between about 8:1 and about 18:1, between about 8:1 and about 10:1, between about 8:1 and about 12:1, between about 98:2 and about 80:20, between about 95:5 and about 80:20, between about 95:5 and about 87:13, between about 95:5 and about 86:14, between about 95:5 and about 85:15, between about 93:17 and about 85:15, between about 90:10 and about 85:15; and the antimicrobial synergistically effective amount is between about 2.5 and 4% w/w.

In some embodiments, the antimicrobial composition comprises niacinamide, and the C4-C15diol; wherein a w/w ratio between niacinamide and C4-C15diol is between about 8:1 and about 20:1, between about 8:1 and about 15:1, between about 8:1 and about 10:1, between about 8:1 and about 12:1, between about 98:2 and about 80:20, between about 95:5 and about 80:20, between about 95:5 and about 87:13, between about 95:5 and about 86:14, between about 95:5 and about 85:15, between about 93:17 and about 85:15, between about 90:10 and about 85:15; and the antimicrobial synergistically effective amount is between about 2.5 and 4% w/w.

In some embodiments, the antimicrobial composition comprises niacinamide, the C4-C15diol, wherein a w/w ratio between niacinamide and C4-C15diol is as described above, and further comprises a hydroxamic acid (e.g. Caprylhydroxamic acid).

In some embodiments, wherein a w/w ratio between the C4-C15diol (e.g. caprylyl glycol, EHG or decanediol) and a hydroxamic acid (e.g. Caprylhydroxamic acid) within the antimicrobial composition is between 1:1 and 5:1, between 2:1 and 5:1, between 2:1 and 4:1, or about 3:1, including any range between.

In some embodiments, a saponin contains a hydrophobic aglycone (e.g. a sterol) and a hydrophilic glycoside (sugar) head group covalently bound to the aglycone. In some embodiments, the saponin is selected from the group consisting of dammaranes, tirucallanes, lupanes, hopanes, oleananes, taraxasteranes, ursanes, cycloartanes, lanostanes, cucurbitanes, solanine, solanidine, tomatine, chaconine, tomatidine and steroids, with or without a linked sugar moiety.

In some embodiments, the saponin is selected from the group consisting of solanine, solanidine, tomatine, chaconine, tomatidine and any combination of same. In some embodiments, the saponin is or comprises a tea saponin. In some embodiments, the saponin is or comprises a plant extract. In some embodiments, the saponin is or comprises an enriched or a purified plant product or a plant extract. In some embodiments, the saponin is or comprises a Camellia sinensis extract (e.g. seed extract).

In some embodiments, the antimicrobial composition of the invention is composed essentially of (i) niacinamide, and/or a salt thereof; and (ii) the cationic surfactant selected from a saponin, Polyquaternium 78, ELA and/or Cola lipid C, or any combination thereof; wherein a weight ratio between (i) and (ii) is between 30:1 and 5:1.

In some embodiments, the antimicrobial composition of the invention is composed essentially of (i) niacinamide, and/or a salt thereof; and (ii) the cationic surfactant selected from a saponin, alkyl polyglucoside-based quaternary ammonium of Formula 1, phosphate-based quaternary ammonium of Formula 2, and guanidine-based cationic surfactant of Formula 3, or any combination thereof; wherein a weight ratio (e.g. antimicrobial synergistically effective amount) between (i) and (ii) is between 30:1 and 5:1.

In some embodiments, the antimicrobial composition of the invention is composed essentially of (i) niacinamide, and/or a salt thereof; and (ii) the cationic surfactant selected from Polyquaternium 78, ELA and/or Cola lipid C, or any combination thereof; wherein a weight ratio between (i) and (ii) is between 30:1 and 5:1.

In some embodiments, the antimicrobial composition of the invention is composed essentially of (i) niacinamide, and/or a salt thereof; and (ii) polylysine; wherein a weight ratio between (i) and (ii) is between 100:1 and 20:1. In some embodiments, the antimicrobial composition of the invention is composed essentially of (i) niacinamide, and/or a salt thereof; (ii) polylysinel and (iii) hexylresorcinol; wherein a weight ratio between (i) and (ii) is between 100:1 and 20; and wherein a weight ratio between (ii) and (iii) is between about 1:2 and 2:1, or about 1:1.

In some embodiments, the antimicrobial composition of the invention is composed essentially of (i) niacinamide, and/or a salt thereof; and (ii) the diol as disclosed above; wherein a weight ratio between (i) and (ii) is between 8:1 and 20:1. In some embodiments, the antimicrobial composition of the invention is composed essentially of (i) niacinamide, and/or a salt thereof; and (ii) the diol; wherein a weight ratio between (i) and (ii) is between 8:1 and 20:1; and wherein the diol consists of glyceryl caprylate and optionally the C4-C15diol selected from decanediol, caprylyl glycol and ethylhexyl glycerin.

In some embodiments, the antimicrobial composition of the invention is composed essentially of (i) niacinamide, and/or a salt thereof; and (ii) glyceryl caprylate and decanediol; wherein a weight ratio between (i) and (ii) is between 8:1 and 20:1; and wherein a weight ratio between glyceryl caprylate and decanediol is about 3:1.

In some embodiments, the antimicrobial composition of the invention is composed essentially of (i) niacinamide, and/or a salt thereof; (ii) the C4-C15diol (e.g. caprylyl glycol, ethylhexyl glycerin, or decanediol) and (iii) Caprylhydroxamic acid; wherein a w/w ratio between (i) and (ii) is between about 8:1 and about 20:1; and wherein a w/w ratio between (ii) and (iii) is about 3:1. In some embodiments, the antimicrobial composition consisting essentially of (i), (ii) and (iii) is characterized by a broad spectrum preservative activity against bacteria fungi and mold.

In some embodiments, the antimicrobial composition comprises the antimicrobial synergistically effective amount of the niacinamide and of the surfactant, as described herein. In some embodiments, the antimicrobial synergistically effective amount comprises a w/w concentration of niacinamide within the formulation (i.e. upon addition thereto of the antimicrobial synergistically effective amount of the antimicrobial composition) of at least about 2%, at least about 2.3%, at least about 2.5%, at least about 2.8%, at least about 3%, at least about 3.5%, at least about 4%, between 2 and 5%, between 2 and 4.5 between 2.5 and 4%, between 2.3 and 3.5%, between 2.5 and 3.5% w/w, including any range between. In some embodiments, the antimicrobial synergistically effective amount comprises a w/w concentration of niacinamide as described hereinabove, and further comprises a w/w concentration of the surfactant (e.g. the cationic surfactant disclosed hereinabove, the diol, polylysine and/or a saponin) at least about 0.03%, at least about 0.05%, at least about 0.07%, at least about 0.1%, at least about 0.15%, at least about 0.2%, at least about 0.25%, at least about 0.3%, at least about 0.5%, between 0.1 and 0.5, between 0.2 and 0.5%, between 0.25 and 0.5%, between 0.25 and 0.4%, between 0.2 and 0.3%, between 0.2 and 0.35% w/w including any range between.

In some embodiments, the antimicrobial composition of the invention comprises (i) between 2 and 80%, between 2 and 70%, between 2 and 60%, between 2 and 50%, between 3 and 80%, between 3 and 70%, between 3 and 50%, between 2 and 10%, between 2 and 20%, between 3 and 10%, between 3 and 30%, between 3 and 40%, between 10 and 80%, between 10 and 60%, between 10 and 50%, between 10 and 30%, between 10 and 20%, between 20 and 80%, between 20 and 60%, between 20 and 50% w/w of niacinamide (including any salt and any antimicrobially active derivative thereof) or any range between, and (ii) between 0.03 and 20%, between 0.05 and 20%, between 0.05 and 15%, between 0.1 and 20%, between 0.1 and 15%, between 0.1 and 10%, between 0.05 and 5%, between 0.1 and 6%, between 0.5 and 5%, between 0.5 and 10%, between 0.05 and 3%, between 0.05 and 2%, between 0.05 and 1.5%, between 0.1 and 3%, between 0.1 and 2%, between 0.1 and 1.5% w/w including any range between of the surfactant (e.g. the cationic surfactant, such as Polyquaternium 78, ELA, and/or Cola lipid C; the diol such as caprylyl glycol, ethylhexyl glycerin and decanediol, or any combination thereof; polylysine and/or a saponin); or wherein the antimicrobial composition of the invention comprises between 10 and 60%, or between 10 and 50% w/w of (i) and between 0.1 and 5% w/w of (ii).

In some embodiments, the antimicrobial composition of the invention further comprises an antimicrobially effective amount of an additional agent selected from a hydroxamic acid and/or a salt thereof (e.g. C5-C30 hydroxamic acid, such as Caprylhydroxamic acid abbrev. as CA), hexylresorcinol, a zinc compound, including any salt or any combination thereof. In some embodiments, the additional agent has an additive and/or synergistic antimicrobial activity when used together with niacinamide and the surfactant disclosed hereinabove.

In some embodiments, the zinc compound comprises Zn(II) cation, and/or a salt thereof. In some embodiments, the zinc compound comprises Zn(II) cation and a counterion, wherein the counterion is any pharmaceutically-, cosmeceutically, and/or food-acceptable counterion. In some embodiments, the zinc compound is or comprises a Zn(II) complex. In some embodiments, the zinc compound comprises zinc acetate, zinc pyrithione, zinc gluconate, zinc lactate, zinc glycinate, zinc chloride, zinc carbonate, zinc phosphate, zinc nitrate, zinc sulfate, or any combination thereof.

In some embodiments, the antimicrobial composition of the invention consists essentially of niacinamide, the surfactant (i.e. the cationic surfactant disclosed hereinabove, the diol, polylysine and/or a saponin) and the additional agent as the sole antimicrobially active agents.

In some embodiments, a w/w concentration of the additional agent within the antimicrobial composition of the invention is at least about 0.01%, at least about 0.03%, at least about 0.05%, at least about 0.1%, at least about 0.2%, at least about 0.3%, at least about 0.5%, including any range between.

In some embodiments, a w/w concentration of the additional agent within the antimicrobial composition of the invention is between about 0.01 and 5%, between about 0.01 and 3%, between about 0.03 and 5%, between about 0.05 and 5%, between about 0.05 and 3%, between about 0.05 and 1.5%, between about 0.05 and 2%, including any range between.

In some embodiments, a w/w ratio between the additional agent and the surfactant within the antimicrobial composition of the invention (wherein the additional agent and the surfactant are as described herein) is between about 0.1:1 and about 1:1, between about 0.2:1 and 1:1, between about 0.2:1 and 0.5:1, between about 0.1:1 and 0.5:1, including any range between.

In some embodiments, a w/w ratio between the additional agent and niacinamide (e.g. synergistically effective amount) within the antimicrobial composition of the invention is between 1:100 and 1:20, between 1:100 and 1:50, between 1:60 and 1:20, between 1:80 and 1:20, between 1:100 and 1:50, between 1:80 and 1:50, between 1:50 and 1:20, including any range between.

In some embodiments, any one of the active agents of the invention is or comprises a pharmaceutically-, cosmetically, and/or food-acceptable compound. In some embodiments, the antimicrobial composition of the invention is composed essentially of pharmaceutically pure compounds, or food-grade compounds.

In some embodiments, the antimicrobial composition of the invention (e.g. antimicrobial composition of the invention) consist essentially of the active ingredients described herein (e.g. (i) niacinamide and/or a salt thereof; (ii) one or more cationic surfactant(s), the diol, polylysine and/or saponin(s) and/or a salt or a combination thereof; and optionally (iii) and one or more additional agent(s) such as CA, hexylresorcinol and/or the zinc compound), wherein the concentration and/or ratio between the active ingredients within the antimicrobial composition of the invention is as described herein.

In some embodiments, the antimicrobial composition is a powderous composition. In some embodiments, the antimicrobial composition is a solid composition. In some embodiments, the antimicrobial composition is in a dry state. In some embodiments, the antimicrobial composition is a solid antimicrobial composition. In some embodiments, the antimicrobial composition is a fluid (e.g. a liquid) at a temperature between 5 and 95° C. In some embodiments, the antimicrobial composition is a semi-solid or a gel at a temperature between 5 and 95° C. In some embodiments, the antimicrobial composition is a flowable composition.

In some embodiments, the antimicrobial composition of the invention comprises the active (or antimicrobial) ingredients as described hereinabove, and further comprises a carrier. In some embodiments, the carrier is a pharmaceutically acceptable carrier, cosmeceutically acceptable carrier, a food grade carrier, or any combination thereof. In some embodiments, the antimicrobial composition of the invention is an aqueous composition. In some embodiments, the aqueous composition is in a form of a solution, emulsion, suspension or a dispersion.

In some embodiments, a w/w concentration of the acceptable carrier within the antimicrobial composition is between 50 and 99%, between 60 and 99%, between 70 and 99%, between 80 and 99%, between 90 and 99%, between 50 and 70%, between 70 and 90%, including any range therebetween.

In some embodiments, the antimicrobial composition of the invention is an aqueous solution comprising the active (or antimicrobial) ingredients substantially or fully dissolved therewithin.

In some embodiments, the antimicrobial composition of the invention is an aqueous composition (e.g. a solution, emulsion/dispersion, etc.) comprising the antimicrobially synergistic effective amount of the active (or antimicrobial) ingredients substantially or fully dissolved therewithin, and wherein a concentration of the active ingredients is as described herein. In some embodiments, the antimicrobial composition of the invention is an aqueous composition and the combined concentration of the active ingredients (i.e. niacinamide, the surfactant and optionally the additional agent, as disclosed herein) is between 10 and 60% w/w, and wherein the aqueous composition optionally comprises a surface active agent (e.g. Coco-glucoside) at a concentration between 0.5 and 5%, between 1 and 5%, including any range between.

In some embodiments, the composition of the invention is a ready to use composition or is in a form of a dilutable concentrate.

In some embodiments, the antimicrobial composition of the invention is in a form of a kit. In some embodiments, the constituents are at a synergistically effective amount within the kit, wherein the synergistically effective amount is as described herein. In some embodiments, the kit comprises any one of the active ingredients described hereinabove, wherein at least a portion or all of the active ingredients are stored in separate container or packages.

In some embodiments, the kit comprises (i) niacinamide, a salt thereof or both; and (ii) the surfactant selected from a cationic surfactant, the diol, polylysine and the saponin; wherein (i) and (ii) are stored in separate containers. In some embodiments, the kit further comprises (iii) the additional agent as disclosed hereinabove.

In some embodiments, the kit is an antimicrobial, sanitizing, sterilizing or a preservative kit. In some embodiments, the kit comprises one or more compartments (e.g. a first compartment, a second compartment, and optionally a third compartment), wherein each of the compartments comprises one or more of the constituents of the kit, as disclosed herein.

In some embodiments, the kit comprises instructions for mixing of (i) the first compartment, (ii) the second compartment, and optionally (iii) the third compartment so as to obtain a mixture, wherein a ratio between niacinamide (i) and (ii) the surfactant selected from a cationic surfactant, the diol, polylysine and the saponin within the mixture is between 100:1 and 5:1, between 30:1 and 8:1, between 20:1 and 8:1, between 20:1 and 5:1, between 50:1 and 8:1, by weight, including any range between, optionally, wherein a w/w ratio between (iii) the additional agent and (ii) within the mixture is between about 0.1:1 and 1:1, between about 0.2:1 and 1:1, between about 0.2:1 and 0.5:1, between about 0.1:1 and 0.5:1, or about 1:3, including any range between. In some embodiments, the mixture is the antimicrobial composition of the invention.

In some embodiments, the kit further comprises instructions for adding a predetermined amount of the mixture to an article (i.e. a liquid, or a semi liquid formulation) to obtain an antimicrobial effective amount of the antimicrobial composition within the article. In some embodiments, the antimicrobial effective amount comprises a combined concentration of the active ingredients of the kit (i.e. i, ii and optionally iii) within the article in a range between about 2 and about 5% w/w, between about 2.5 and about 3% w/w, between about 2.5 and about 4% w/w, between about 3 and about 4% w/w, between about 3 and about 5% w/w, between 2.5 and 3.5% w/w, including any range between.

In some embodiments, the antimicrobial effective amount comprises (i) a concentration of niacinamide and/or a salt thereof of between about 2 and about 5% w/w, between about 2.5 and about 3% w/w, between about 2.5 and about 4% w/w, between about 3 and about 6% w/w, between about 3 and about 5% w/w, between about 3 and about 4% w/w; (ii) a concentration of the cationic surfactant (e.g. any one of Polyquaternium 78, ELA, Cola lipid C and saponin); of one or more diol(s), or of polylysine between about 0.03 and about and 1% w/w, between about 0.05 and about and 1% w/w, between about 0.07 and about and 1% w/w, between about 0.1 and about and 1% w/w, between about 0.15 and about and 1% w/w, between about 0.15 and about 0.5% w/w, between about 0.1 and about 0.5% w/w, between about 0.1 and about 0.8% w/w, between about 0.1 and about 0.2% w/w, between about 0.15 and about 0.3% w/w, between about 0.15 and about 0.4% w/w, between about 0.15 and about 0.6% w/w; and optionally (iii) a concentration of the additional agent between about 0.01 and about 0.2% w/w, between about 0.01 and about 0.1% w/w, between about 0.03 and about 0.1% w/w, between about 0.02 and about 0.1% w/w, between about 0.03 and about 0.05% w/w, between about 0.03 and about 0.07% w/w, between about 0.05 and about 0.1% w/w, between about 0.05 and about 0.2% w/w, wherein the concentration refers to the concentration of the active agent in the article (e.g. a cosmetic formulation, a pharmaceutical formulation or a food product).

In some embodiments, the terms “constituents”, “active ingredients”, and “preservative” including any grammatical from thereof, are used herein interchangeably.

In some embodiments, the kit comprises instructions for dilution and optionally for mixing of the first compartment, the second compartment, so as to obtain the antimicrobial composition of the invention comprising a synergistically effective amount of the constituents. In some embodiments, the kit comprises instructions for dilution and optionally for mixing of the first compartment, the second compartment, and optionally the third compartment, so as to obtain the antimicrobial composition of the invention comprising a synergistically effective amount of the constituents.

In some embodiments, the first compartment, the second compartment and the third compartment of the kit are mixed simultaneously. In some embodiments, the first compartment, the second compartment and the third compartment of the kit are mixed subsequently. In some embodiments, the first compartment, and the second compartment and optionally the third compartment of the kit are applied simultaneously or subsequently. In some embodiments, mixing comprises dosing the compartments in an amount sufficient for obtaining a predetermined molar ratio of the to the active agents within the antimicrobial composition.

In some embodiments, the first compartment, the second compartment and the third compartment of the kit are diluted before mixing.

In some embodiments, dosing comprises dispensing a predetermined amount of the first compartment, a predetermined amount of the second compartment, a predetermined amount of the third compartment and subsequent mixing thereof. In some embodiments, dosing comprises dispensing a predetermined amount of the first compartment, a predetermined amount of the second compartment, so as to obtain a predetermined concentration of the constituents within the antimicrobial composition, wherein the predetermined concentration refers to the antimicrobial effective amount, as described hereinabove.

In some embodiments, the antimicrobial composition or the kit of the invention is devoid of an antimicrobial agent (such as a preservative) which is not any one of the active ingredients disclosed herein. In some embodiments, the antimicrobial composition or the kit is devoid of phenoxyethanol. In some embodiments, the antimicrobial composition or the kit is devoid of benzyl alcohol. In some embodiments, the antimicrobial composition or the kit is devoid of a paraben.

In some embodiments, the antimicrobial composition of the invention is characterized by an antimicrobial activity (e.g. sterilizing or preservation activity) within the article or on a surface of a substrate upon contacting an antimicrobial effective amount of the antimicrobial composition with the article or with the surface under suitable conditions (such as a temperature between 10 and 40° C., for a time period between 1 minute and 14 days, including any range between). In some embodiments, antimicrobial activity is as described hereinbelow.

In some embodiments, the article and/or the surface refers to article or surface prone of microbial infestation (such as articles comprising a water content of at least 10%, at least 50%, or between 10 and 99%, between 20 and 90%, between 20 and 80%, between 30 and 99%, including any range between).

In some embodiments, the antimicrobial composition or the kit of the invention is an antimicrobial preparation formulated for use in an article or on a surface susceptible to microbial growth as a preservative. In some embodiments, the article is selected from a cosmetic article, a pharmaceutical article, and/or a food-grade article. In some embodiments, the antimicrobial preparation is selected from a solid formulation, a liquid formulation (e.g. an aqueous formulation), a gel, an emulsion, a cream, a foam, or any combination thereof.

In some embodiments, the antimicrobial preparation comprises the antimicrobial composition of the invention and further comprises an additive, such as thickener, fragrance, a buffering agent, a stabilizer, an antioxidant, etc.

In some embodiments, the antimicrobial preparation further comprises a surfactant (e.g. Coco glucoside) which is not an antimicrobial or a synergistic surfactant, as disclosed herein. In some embodiments, a w/w concentration of the surfactant within the formulation is between 0.1 and 10%, between 0.1 and 0.5%, between 0.5 and 10%, between 1 and 10%, between 0.5 and 1%, between 1 and 3%, between 3 and 5%, between 5 and 10%, including any range therebetween.

In some embodiments, the surfactant is selected from an anionic surfactant, a cationic surfactant, and a non-ionic surfactant, including any combination thereof.

In some embodiments, the antimicrobial preparation is an anionic formulation, cationic formulation, zwitterionic formulation, or nonionic formulation.

Non-limiting examples of surfactants include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically, cosmeceutically and/or food acceptable salts of fatty acids such as stearic, oleic, palmitic, and lauric acids), cationic surfactants (e.g. pharmaceutically, cosmeceutically and/or food acceptable quaternary ammonium salts such as benzalkonium chloride, benzethonium chloride, and cetylpyridinium chloride) or any combination thereof. Additional non-limiting examples of anionic surfactants include but are not limited to: (C₆-C₈)alkyl-sulfate and/or sulfonate (e.g., sodium or potassium lauryl sulfate, sodium or potassium dodecyl sulfate), fatty alcohol ether sulfate salt (e.g., (C₁₂-C₁₄)alkyl-O—(CH₂CH₂O)₂—SO₃⁻, ZOHARPON ETA 27), polyacrylate (e.g., sodium or potassium polyacrylates), or any combination thereof. Additional non-limiting examples of non-ionic surfactants include but are not limited to: alkyl-polyglycoside (e.g., Triton CG 110, APG 810), polyethyleneglycol-(C₁₁-C₁₅)alkyl-ether (such as Imbentin AGS/35), alkoxylated fatty alcohol (such as Plurafac LF221), or any combination thereof. In some embodiments, the nonionic surfactant is or comprises Decyl glucoside. In some embodiments, the cationic surfactant is or comprises guar hydroxypropyl trimonium chloride. In some embodiments, the anionic surfactant is or comprises Disodium Cocoamphodiacetate.

In some embodiments of the present invention, the antimicrobial preparation further comprises a buffer solution or a pH adjuster to control the desired pH of the formulation.

The antimicrobial preparation of the invention can be prepared by any commonly used method for preparing a composition of materials. For example, the components of the antimicrobial preparation may be added and mixed together, or one of the components may be added to the other in the form of a solution which may, if desired, be evaporated or lyophilized after mixing for obtaining a homogeneous and stable solution or suspension.

### Articles Comprising the Composition of the Invention

According to some embodiments of the present invention there is provided an article comprising an antimicrobial affective amount of the antimicrobial composition of the invention, as described herein. In some embodiments, the article further comprises an acceptable carrier (e.g, wherein the acceptable carrier is selected from a cosmetically acceptable carrier, pharmaceutically acceptable carrier, and a nutraceutically acceptable or a food-grade carrier). In some embodiments, the article comprises the antimicrobial composition of the invention as a preservative or as an antimicrobial agent. In some embodiments, the article comprises the antimicrobial composition of the invention as the sole preservative or as the sole antimicrobial agent. In some embodiments, the article is substantially devoid of an additional preservative or an additional antimicrobial agent, other than the antimicrobial composition of the invention.

In some embodiments, the article is selected from a cosmeticarticle (e.g. a cream, ointment, foam, solution, lotion, gel, shampoo, soap, show gel, cleansing solution, etc.) and a personal care product.

Some embodiments of this aspect of present embodiments are included hereinabove, under “Composition” or “The Formulation”, and form an integral part of embodiments relating to “Articles comprising the Formulations”.

According to an aspect of some embodiments of the present invention, there is provided a pharmaceutical, cosmetic or cosmeticproduct comprising the formulation described in any of their respective embodiments herein, for use in treating a medical, cosmetic or cosmeticcondition, as described herein.

According to an aspect of some embodiments of the present invention, the formulation described in any of their respective embodiments herein is used as, or a part of, a preservative in any pharmaceutical, cosmetic or cosmeticproduct or in any article as describe herein.

As used herein, “preservative” is used to prevent the growth of bacteria, fungi and/or molds in any personal care composition or formulation.

According to a further aspect of some embodiments of the present invention, there is provided a use of the formulation described herein in the manufacture of a pharmaceutical, cosmetic or cosmeticproduct, which can be used in treating a medical, cosmetic or cosmeticcondition, as described herein.

In some embodiments, there is provided a method of treating a medical, cosmeticor cosmetic condition treatable by a topical or transdermal administration, the method comprising topically applying the formulation described herein (e.g., in the context of a pharmaceutical, cosmetic or cosmeticproduct) to a skin or mucosal tissue of a subject afflicted by the condition.

The phrases “topical” “topical administrations” and or any grammatical derivative thereof, is meant to encompass applications, which include, without limitation, dermal applications, ophthalmic application, vaginal application, rectal application and intranasal application.

Medical, cosmetic or cosmeticconditions that can benefit from containing the formulations described herein when applied topically, with or without an additional active ingredient, include, but are not limited to, infections caused by pathogenic microorganisms, as discussed in further detail hereinbelow, wounds, particularly when associated with an infection, acne, skin infections, viral blisters such as one caused by herpes, sexual dysfunction such as erectile dysfunction.

Hence, according to some embodiments of the present invention, the pharmaceutical, cosmetic or cosmeticarticle, as described herein, further comprises an antimicrobial agent, as an additional pharmaceutically active agent.

Microbial infections include any infection caused by a pathogenic microorganism, including, bacterial infection, fungal infection, protozoal infection, viral infection and the like, e.g., molluscum contagiosum (a viral infection of the skin or occasionally of the mucous membranes), fungal nail infections, and cutaneous leishmaniasis.

Topical bodily sites include skin, mucosal tissue, eye, ear, nose, mouth, rectum and vagina.

In some embodiments, there is provided an article (e.g., a medical device such as a bandage or adhesive patch), a formulation, or a product, as described herein, configured for topical application, whereby a condition treatable by such as article, product or formulation is an infection caused by a microorganism.

In some embodiments of the present invention, the article is e.g., a fabric, a bandage, a wipe (e.g., a wet wipe), a pledget, a swab, a suppository, a dressing, a solution, a mousse, a pad, or a patch.

In some exemplary embodiments of the present invention, the article is in the form of a solution, a paste, a cream, a lotion, a foam, a gel, an emulsion, an ointment, or a soap.

In some embodiments, the personal care formulation of the present invention can be used to treat skin tissue or on damaged or unhealthy skin tissue.

The phrase “damaged or unhealthy skin tissue” as used herein refers to a deviation from healthy functional skin tissue. In the case of skin-a skin that is weaker, less clastic, and is more prone to injury than healthy skin. The structure of unhealthy or damaged skin is inferior to that of healthy skin (for example, the dermis and epidermis contain fewer cells and collagen).

The phrase “healthy skin tissue” as used herein refers to skin that is strong, elastic, smooth and plump. One purpose of treating healthy skin is to prevent deterioration of skin induced by aging or environmental stress including, but not limited to, microbial infection.

The term “damaged” as used herein, or any grammatical derivative thereof, refers broadly to injuries to the skin and subcutaneous tissue as well as internal organs initiated in any one of a variety of ways (e.g., pressure sores from extended bed rest, wounds induced by trauma, wounds received during or following a surgical procedure and the like) and with varying characteristics. Examples include, but are not limited to, bruises, scrapes, burn wounds, sunburn wounds, incisional wounds, excisional wounds, surgical wounds, necrotizing fascitis, ulcers, venous stasis ulcers, diabetic ulcers, decubitus ulcers, aphthous ulcers, pressure ulcers, scars, alopecia areata, dermatitis, allergic contact dermatitis, atopic dermatitis, berloque dermatitis, diaper dermatitis, dyshidrotic dermatitis, psoriasis, eczema, crythema, warts, anal warts, angioma, cherry angioma, athlete's foot, atypical moles, basal cell carcinoma, Bateman's purpura, bullous pemphigoid, Candida, chondrodermatitis helicis, Clark's nevus, cold sores, condylomata, cysts, Darier's disease, dermatofibroma, Discoid Lupus Erythematosus, nummular eczema, atopic eczema, dyshidrotic eczema, hand eczema, Multiforme Erythema Nodosum, Fordyce's Condition, Folliculitis Keloidalis Nuchac, Folliculitis, Granuloma Annulare, Grover's Disease, heat rash, herpes simplex, herpes zoster (shingles), Hidradenitis Suppurativa, Hives, Hyperhidrosis, Ichthyosis, Impetigo, Keratosis Pilaris, Keloids, Keratoacanthoma, Lichen Planus, Lichen Planus Like Keratosis, Lichen Simplex Chronicus, Lichen Sclerosus, Lymphomatoid Papulosis, Lupus of the Skin, Lyme Disease, Lichen Striatus, Myxoid Cysts, Mycosis Fungoides, Molluscum Contagiosum, Moles, Nail Fungus, Necrobiosis Lipoidica Diabeticorum, Nummular Dermatitis, Onychoschizia, Onychomycosis, Pityriasis Lichenoides, Pityriasis Rosea, Pityriasis Rubra Pilaris, Plantar Warts, Poison Ivy, Poison Oak, Pompholyx, Pseudofolliculitis Barbae, Pruritus Ani and Pityriasis Alba.

In some embodiments, the article is in a form of a cationic formulation, an anionic formulation, or a non-ionic formulation.

In some embodiments, a cationic formulation comprises about 10%, about 5%, about 4%, about 3%, by weight, of the antimicrobial composition or of the active ingredients as described herein, including any value therebetween. Each possibility represents a separate embodiment of the invention. In some embodiments, a cationic formulation further comprises between 0.1 and 20%, between 0.1 and 1%, between 0.1 and 10%, between 0.1 and 5%, between 0.5 and 1%, between 0.5 and 10%, between 0.5 and 5%, between 1 and 20%, between 1 and 10% by weight of a cationic surfactant, including any range between.

In some embodiments, an anionic formulation comprises about 10%, about 5%, about 4%, about 3%, by weight, of the antimicrobial composition or of the active ingredients as described herein, including any value therebetween. Each possibility represents a separate embodiment of the invention.

In some embodiments, a nonionic formulation comprises about 10%, about 5%, about 4%, about 3%, by weight, of the antimicrobial composition or of the active ingredients as described herein, including any value therebetween. Each possibility represents a separate embodiment of the invention.

In some embodiments, the article is a personal care composition.

As used herein, the term “physiologically acceptable” means approved by a regulatory agency of the Federal or a state government or listed in the U.S. Pharmacopeia or other generally recognized pharmacopeia for use in animals, and more particularly in humans.

As used herein, the term “excipient” refers to an inert substance added to a formulation as described herein to further facilitate processes and administration of the active ingredients.

In some embodiments, the disclosed composition, in any embodiment thereof, is a stable formulation.

As used herein the terms “stable formulation”, or “long-lasting formulation”, mean that the formulation remains in a state or condition of sufficient stability to have utility as a personal care agent, while maintaining the antimicrobial activity (with ±20% variation). For example, and without limitation, the formulation has a sufficient stability to allow storage at a convenient temperature, e.g., between 10° C. and 30° C., under ambient atmosphere (normal atmospheric pressure, atmospheric gases, etc.) for a reasonable period of time of e.g., longer than one month, longer than three months, longer than six months, and longer than one year or between 1 week and 1 y, between 1 w and 2 y, including any range between. In some embodiments, the stable formulation retains its initial chemical composition, active ingredients content (with ±20% variation) and/or retains it's physical appearance or a physical state when stored at convenient temperature, e.g., between 10° C. and 30° C., under ambient atmosphere (normal atmospheric pressure, atmospheric gases, etc.) for a reasonable period of time such as up to 2 years.

In some embodiments, the disclosed composition is incorporated into a substrate (such as a textile substrate, a moist textile substrate, a moist substrate, an edible matter, a plastic substrate, and/or a formulation such as a solid formulation, a liquid formulation, a gel, an emulsion, a foam, or any combination thereof) susceptible to microbial growth as a preservative (or an antimicrobial composition).

In some embodiments, the disclosed composition is incorporated in a preservative or in a personal care system in a concentration (by total weight) of e.g., 0.1%, 0.5%, 1%, 1.5%, 2%, 2.5%, 3%, 3.5%, 4%, 4.5%, 5%, 5.5%, 6%, 6.5%, 7%, 6.5%, 8%, 8.5%, 9%, 9.5%, 10%, 10.5%, 11%, 11.5%, 12%, 12.5%, 13%, 13.5%, 14%, 14.5%, or 15%, including any value and range therebetween. Each possibility represents a separate embodiment of the invention.

As used herein, the phrase “personal care” refers to compositions that can be formulated in various cosmetic and pharmaceutical consumer products utilizing a variety of delivery systems and carrier bases. Such consumer product forms include, but are not limited to, fabric, a bandage, a wipe, a baby wipe, a pledget, a swab, a suppository, shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

### Antimicrobial Activity

According to an aspect of some embodiments of the present invention there is provided a method of inhibiting or reducing or retarding the formation of load of a microorganism and/or the formation of a biofilm, in and/or on the article, as disclosed herein. In some embodiments, the method comprises contacting the article with a synergistically effective amount of any one of the antimicrobial composition disclosed herein, wherein the synergistically effective amount is sufficient for obtaining the antimicrobial synergistically effective amount of the antimicrobial composition within the article, and wherein the antimicrobial synergistically effective amount is as described hereinabove. In some embodiments, the method comprises contacting the article with any one of the antimicrobial compositions disclosed herein. In some embodiments, the method comprises contacting the article with an antimicrobial effective amount of the antimicrobial composition of the invention.

In some embodiments, the method comprises incorporating in and/or on the article the antimicrobially effective amount of any one of the antimicrobial composition s disclosed herein, including any of the respective embodiments thereof. In some embodiments, the method comprises incorporating in and/or on the article any one of the formulations disclosed herein, including any of the respective embodiments thereof. The article can be any one of the articles described herein.

According to an aspect of some embodiments of the present invention there is provided a method of preserving a cosmetic product, comprising adding to the cosmetic product an antimicrobially effective amount of any one of the antimicrobial compositions disclosed herein.

According to an aspect of some embodiments of the present invention there is provided a method of preserving a personal care product, comprising adding to the personal care product antimicrobially effective amount of any one of the antimicrobial composition s disclosed herein.

In some embodiments, the components in the formulation act in synergism.

In some embodiments, the term synergism, or any grammatical derivative thereof, is defined as the simultaneous action of two or more compounds in which the total response of an organism to the combination is greater than the sum of the individual components. Although many combinations of antimicrobial compounds have been studied, a synergistic effect is rarely revealed and the global use of antimicrobial combinations with synergistically enhanced activity is rather limited.

Herein “antimicrobial activity” is referred to as an ability to inhibit (prevent), reduce or retard bacterial growth, fungal growth, biofilm formation or eradicate living bacterial cells, or their spores, or fungal cells or viruses in a suspension or in a moist environment.

In some embodiments, the “antimicrobial activity” refers to the ability of the antimicrobial composition to reduce a load of at least one pathogen (expressed in CFU or CFU/ml) in the liquid or the semi-liquid formulation by at least 10, at least 20, at least 50, at least 100 fold, including any range between, two days post application of the antimicrobial synergistically effective amount of the antimicrobial composition. In some embodiments, the “antimicrobial activity” refers to the ability of the antimicrobial composition to reduce a load of pathogen(s) according to US Pharmacopeia and/or E Pharmacopeia guidance.

Herein, inhibiting or reducing or retarding the formation of load of a microorganism refers to inhibiting, reducing, or retarding growth of microorganisms and/or eradicating a portion or all of an existing population of microorganisms. Thus, formulations described herein can be used both in reducing the formation of microorganisms on or in an article, and in killing microorganisms in or on an article or a living tissue.

The microorganism can be, for example, a unicellular microorganism (prokaryotes, archaea, bacteria, eukaryotes, protists, fungi, algae, molds, yeast, euglena, protozoan, dinoflagellates, apicomplexa, trypanosomes, amoebae and the likes), or a multicellular microorganism.

In some embodiments, the microorganism comprises bacterial cells of bacteria such as, for example, Gram-positive and Gram-negative bacteria.

In some embodiments, the Gram-positive bacteria are Staphylococcus aureus, Staphylococcus epidermidis, and Bacillus cereus.

In some embodiments, the Gram-negative bacteria are Escherichia coli, Pseudomonas aeruginosa, and Burkholderia cepacia.

In some embodiments of the present invention, the fungi is Candida albicans.

In some embodiments, the mold is Aspergillus brasiliensis.

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaca, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm may be physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The term “antibiofilm” refers to the capacity of a substance to disturb the formation of a biofilm of bacterial, fungal and/or other cells, and/or to affect a reduction in the rate of buildup of a biofilm of bacterial, fungal and/or other cells, on a surface of a substrate.

As used herein, the term “preventing” in the context of antimicrobial, indicates that the growth rate of the microorganism cells is essentially nullified or is reduced by at least 20%, at least 30%, at least 40%, at least 50%, at least 60%, at least 70%, at least 80%, at least 90%, including any value therebetween, of the appearance of the microorganism in a comparable situation lacking the presence of the antimicrobial formulation of the invention or an article containing same. Each possibility represents a separate embodiment of the invention. Alternatively, preventing means a reduction to at least 15%, 10%, or 5% of the appearance of the microorganism cells in a comparable situation lacking the presence of the formulation or an article containing same. As used herein, the term “reducing” in the context of antimicrobial, indicates that the growth rate (and or microbial loading expressed in CFU or CFU/ml) of the microorganism (including spores, cells, or biofilm thereof) is essentially reduced as compared to a similar article devoid of the antimicrobial formulation or composition of the invention. In some embodiments, the term “essentially reduced” comprises at least 2 times, at least 5 times, at least 10 times, at least 50 times, at least 100 times, at least 1000 times, at least 10.000 times, at least 100.000 times, at least 1000.000 times, between 10 and 1000.000 times, between 100 and 1000.000 times, between 1000 and 10.000.000 times, between 1000 and 1000.000 times, between 10.000 and 1000.000 times CFU/ml reduction, including any range between, as compared to the CFU/ml content of a similar article devoid of the antimicrobial formulation or composition of the invention (also referred to herein as the initial microbial loading). In some embodiments, the antimicrobial composition substantially prevents microbial infestation of the article. In some embodiments, the article comprising the antimicrobial composition of the invention maintains its sterility upon exposing thereof to ambient conditions (e.g. ambient atmosphere comprising one or more microbes) for a time period between 1 day and 2 months, between 1 day and 1month, between 1 and 20d, between 1 and 60d, between 1 and 50d, between 10 and 60d, between 1 and 10d, between 10 and 50d, between 10 and 40d, between 10 and 30d, including any range between. The term “sterility” as used herein, refers inter alia to the microbial load of a formulation or article according to the sterility requirements as recorded in US Pharmacopoeia, or in European Pharmacopoeia, respectively.

Methods for determining a level of appearance of a microorganism cells are known in the art.

Such articles take advantage of the improved antimicrobial activity exhibited by the formulations as described herein.

An article, according to these embodiments, can be also a living tissue, for example, a skin or mucosal tissue, as described herein.

In the context of the present embodiments, the formulations, articles and methods described herein may be used to produce cell inhibiting surface, or a microbial cell killing surface, that remains active for extended periods. Such an antimicrobial surface may not need an additional treatment with antimicrobial compositions, clean-up treatments to effect decontamination and cosmetic painting, thereby simplifying upkeep of the physical condition and appearance of microbial infestation prone surfaces. It is contemplated that in some embodiments, the formulations of the present invention may be easily applied to susceptible surfaces in advance of and/or during exposure to a microbial organism.

### General

As used herein the term “about” refers to +10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Materials and methods

### Microorganisms Strains and Growth Conditions

Escherichia coli (ATCC 8739), Staphylococcus aureus (ATCC 6538), P. aeruginosa (ATCC 9027), Candida albicans (ATCC 10231), and Aspergillus brasiliensis (ATCC 16404) were obtained from ATCC and cultured according to the manufacturer's instructions. These microorganisms are routinely used for minimum inhibitory concentration (MIC), checkerboards and challenge tests according to the US and EU recommendations for the efficacy of preservatives in the cosmetic industry. Bacterial strains were maintained in tryptic soy agar (TSA) (Hy-labs, Rehovot, Israel), while yeast and mold strains were maintained in sabouraud dextrose agar (SDA) (Hy-labs, Israel), supplemented with oxytetracycline at a concentration of 1% (Hy-labs, Israel). Preservative efficacy assays (challenge tests) were performed in order to enumerate the number of microorganisms present in a formulation containing the preservative system, while the diluent solution was supplemented with neutralizers containing 4% Tween 20 and 0.5% lecithin (Hy-labs, Israel).

Minimum inhibitory concentration (MIC) and Checkerboard assay

The minimum inhibitory concentration (MIC in 96 well plates) of the tested compounds and/or combinations thereof were evaluated using a microdilution broth assay against the five pharmacopcia strains. Briefly, growth of E. coli, S. aureus, P. aeruginosa, P. gergovica, B. cepacia, C. albicans, and A. brasiliensis was observed during incubation with two-fold serial dilutions of the abovementioned compounds in Mueller Hinton (MH) broth, using a 96-well plate (BIOFUL, Israel). For the checkerboard assay using a 96-well plate, decreasing concentrations of one compound were added from top to bottom of the plate using serial dilutions. Decreasing concentrations of the second compound were added to the plate from the right, having the highest concentration, to the left side of the plate with the lowest concentration. In both MIC and checkerboard assays, wells were inoculated with 100 μl of test cultures with a final inoculum of 5×10⁵ cfu/mL of bacteria and 5×10³ cfu/mL of fungi. Bacteria inoculated plates were incubated for 24 h at 32° C. with shaking, while fungi inoculated plates were incubated at 23° C. for 48-72 h with shaking. Microorganism growth was evaluated by a spectrophotometer O.D. reads (BioTek, Israel).

### MIC Method in Test Tubes

In additional exemplary procedure, MIC evaluation test with Candida albicans was performed in test tubes. Growth in this case is visually evaluated. A pure culture of a single microorganism was grown in Tryptic soy broth, or Sabouraud dextrose broth for yeast and mold.

In another exemplary procedure, MIC evaluation test with A. brasiliensis was performed in test tubes. Growth in this case is visually evaluated. A pure culture of a single microorganism was grown in Tryptic soy broth, or Sabouraud broth for yeast and mold.

The culture is standardized using standard microbiological techniques to have a concentration of very near 1 million cells per milliliter.

The antimicrobial agent is diluted a number of times, with Tryptic soy broth or Sabouraud broth for fungi. After the antimicrobial agent had been diluted, a volume of the standardized inoculums was added to each dilution vessel, bringing the microbial concentration to approximately 1,000,000 cells per milliliter. The inoculated, serially diluted antimicrobial agent was incubated at an appropriate temperature for the test organism, 24 hours for bacteria 48 for yeast and 72 for mold.

After incubation, the series of dilution vessels was observed for microbial growth, usually indicated by turbidity and/or a pellet of microorganisms in the bottom of the vessel. The last tube in the dilution series that does not demonstrate growth corresponds with the MIC of the antimicrobial agent.

The microorganism strains used for the test were E. coli ATCC 8739, P. aeruginosa ATCC 9027, S. aureus ATCC 6538, C. albicans ATCC 10231, A. brasiliensis ATCC 16404.

### Synergistic Effect

Measuring synergy between preservatives can be done by the widely described Fractional Inhibitory Concentration Index (FICI). While the MIC is defined as the lowest concentration of molecule alone or in combination that inhibited visible growth, the in vitro interactions can be quantified as FICI and calculated using the following formula:

$$FICI = \frac{MIC of compound {A}_{c o m b i n e d}}{MIC of compound {A}_{a l o n e}} + \frac{MIC of compound {B}_{c o m b i n e d}}{MIC of compound {B}_{a l o n e}}$$

FICI values were interpreted as follows: <0.5 synergy, 0.5-0.75 partial synergy, 0.75-1.0 additive effect 1.0-4.0 no interaction, and a FICI of >4.0 was defined as antagonism.

### Challenge Test

Challenge tests of preservative efficacy in formulations were performed according to the ISO 11930 regulations. The total count for any prior contamination was performed for all formulations. Specifically, in order to evaluate the antimicrobial and antifungal activity of the preservative system, samples were inoculated separately with each microorganism at a final concentration of 106 cfu/mL for bacteria and 10⁵ cfu/mL for yeast and mold. Samples were incubated in the dark at 22° C. for 28 days. The preservative efficacy was determined by sampling 1 g from the formulation at each time-point of 2, 7, 14, 21, and 28 days. To enumerate the microorganisms at each time point, serial dilutions were made up to 10-4, and 1 mL were seeded in duplicates onto a petri dish with the appropriate media TSA/SDA (bacteria vs. yeast and mold, respectively), using the pour plate method. Plates were incubated at 32° C. for three days for bacteria while yeast and mold were incubated at 22° C. for five days until the enumeration of viable microorganisms.

The nonionic basic cream and anionic shampoo formulations are shown in Table 1.

TABLE 1

| Nonionic- Leave on | | Anionic-Rinse off | | Cationic Mask | |
|---|---|---|---|---|---|
| basic cream | | shampoo | | Rinse-off | |
| Water | q.s. | Water | q.s. | Water | q.s. |
| Glycerin | 4% | Glycerin | 0.3% | Glycerin | 3% |
| Cetearyl | 5% | Cocamidopropyl | 1% | Cetearyl | 2% |
| Glucoside (and) | | Betaine | | Glucoside (and) | |
| Cetearyl alcohol | | | | Cetearyl alcohol | |
| Caprylic Capric | 3% | Cocamide DEA | 1% | Caprylic Capric | 3% |
| Triglycerides | | | | Triglycerides | |
| Cetearyl Alcohol | 4.8% | SLES 70% | 15% | Cetearyl Alcohol | 5% |
| (and) Ceteareth-20 | | | | (and) Ceteareth-20 | |
| Tocopheryl | 0.1% | Citric acid | 0.07% | Behentrimonium | 1% |
| acetate | | | | Chloride | |
| Preservative | 2.5-3% | NaCl | 3.5% | Preservative | 2.5-3% |
| Citric acid | q.s. | Preservative | 2.5-3% | pH | 6-7 |
| pH | 6-7 | pH | 6-7 | Water | q.s. |

### Example 1

### Preservative Properties

First the inventors tested antimicrobial effect of a combined application of 0.8% w/w of niacinamide and 0.1% of a cationic surfactant disclosed herein (e.g. Cola Lipid C, and/or Polyquaternium 78). The inventors observed sufficient antibacterial activity of the tested formulations (e.g. when applied to a basic cream formulation), however, the formulations showed only about 1000 times reduction of the initial fungal load (A. brasiliensis). Accordingly, the inventors aimed to optimize the formulation to induce even greater reduction of the initial fungal loading.

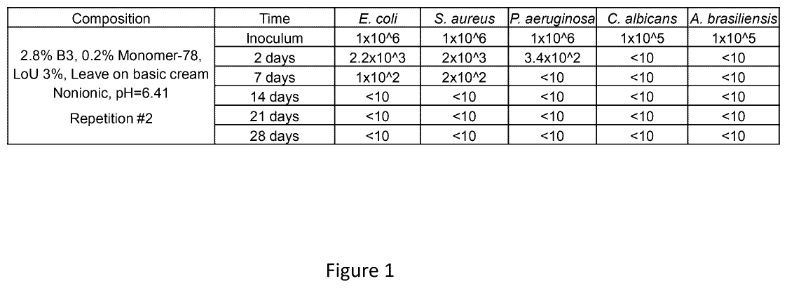

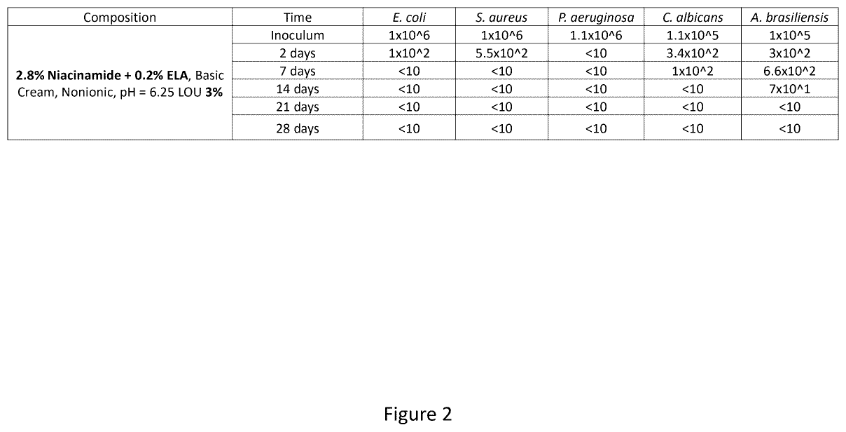

Upon extensive experimentations, the inventors found that a combination of about 2.8% w/w niacinamide with about 0.2% of the cationic surfactant disclosed herein (e.g. Cola Lipid C, Polyquaternium 78 and/or ELA) results in a broad-range antimicrobial activity against tested bacteria and fungi (see FIGS. 1-2 presenting between about 104 and about 106 times reduction of the initial microbial loading). Combined administration of 2.8% w/w niacinamide with 0.2% ELA resulted in efficient reduction of all the tested microbial, thus conforming with the EP acceptance criteria.

Of note, cationic surfactant or niacinamide showed only limited antimicrobial activity at the above mentioned concentrations. Only a combination of both niacinamide and at least one cation surfactant resulted in a broad range antimicrobial activity against the tested fungal and bacterial species (see FIGS. 1-2).

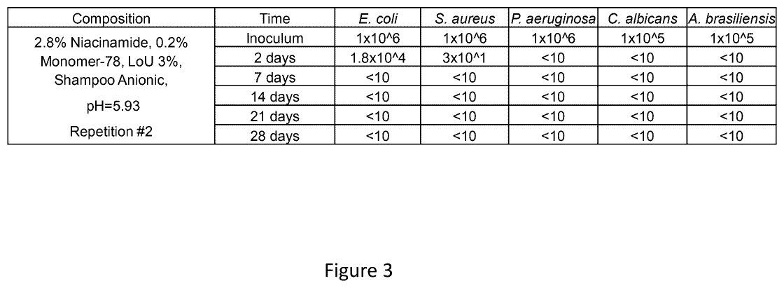

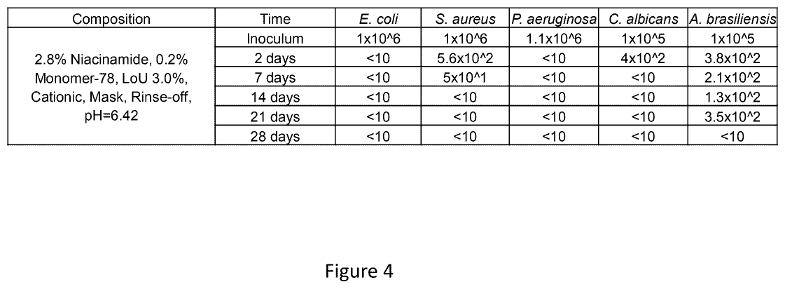

Further, the preservative effectiveness was successfully demonstrated in different formulations such as nonionic basic cream, anionic shampoo and cationic formulations (see FIGS. 3-4).

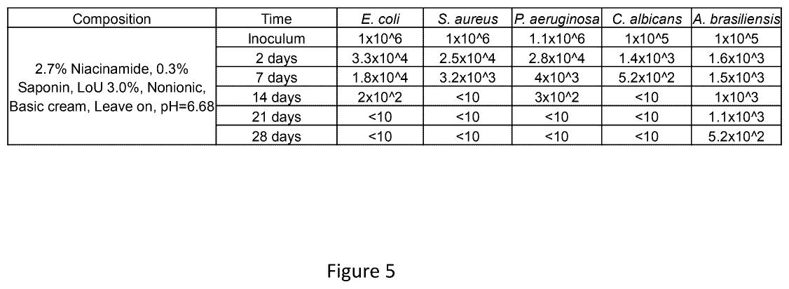

Additionally, the inventors found a synergistic antimicrobial efficiency of niacinamide in combination with saponin. Exemplary formulations (e.g. non-ionic basic cream) containing about 2.7% niacinamide and about 0.3% of a saponin (Camellia sinensis seed extract based saponin) showed a broad spectrum antimicrobial activity, as presented by FIG. 5. The inventors postulate that increased concentration of saponin in the final formulation or article will result in an enhanced antimicrobial activity (especially against fungi such a Aspergillus species).

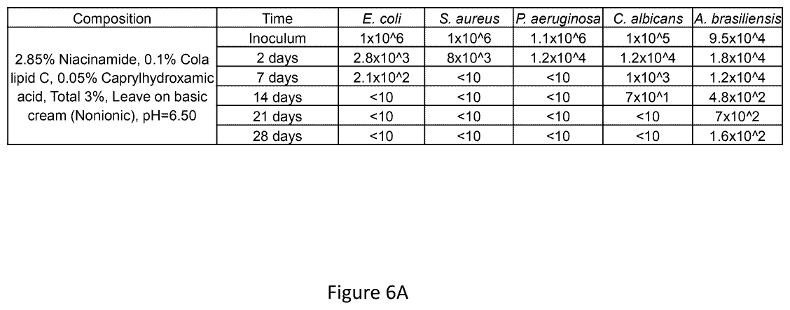

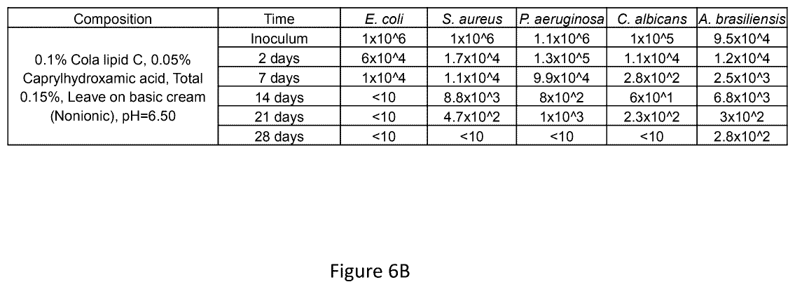

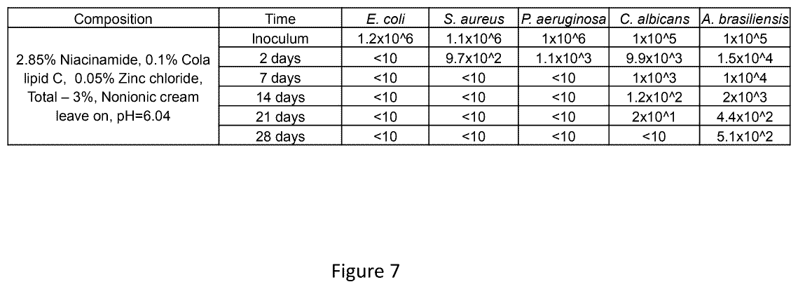

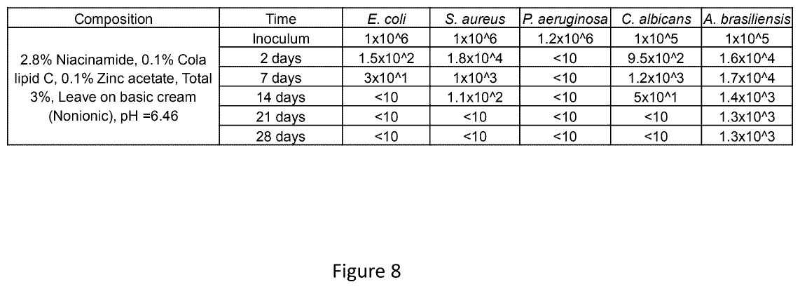

Furthermore, the inventors obtained improved efficacy e.g. almost complete eradication (or at least 4-6 orders of magnitude reduction of the initial CFU) of microbes including bacteria and fungi from the inoculated formulations using only about 0.05% w/w of an additional agent such as a zinc (II) salt, or Caprylhydroxamic acid together with the same concentration of niacinamide and only 0.1% of the cationic surfactant (see FIGS. 6A, 7 and 8).

The results of the combined application of niacinamide, ColaLipid C and Caprylhydroxamic acid, as compared to a control (e.g. a similar formulation devoid of niacinamide) are presented in FIGS. 6A-B.

Thus, it is presumed that the above mentioned synergistic compositions can be successfully implemented at a minimum total antimicrobial effective concentration of about 3-4% w/w.

### Example 2

The inventors have tested a potential preservative activity of different combinations including niacinamide and various diols (w/w ratio between niacinamide and a diol of ˜19:1). The results of these test are summarized in Table 2 below showing MIC of the tested combinations. Of note, the MIC of niacinamide (as the sole representative) was between about 15000 and 30000 for all the tested microorganisms.

TABLE 2

| Preservative | E. coli | S. aureus | P. aeruginosa | C. albicans | A. brasiliensis |
|---|---|---|---|---|---|
| Niacinamide + | 3000 | 3000 | 3000 | 3000 | 3000 |
| Propanediol | | | | | |
| Niacinamide + | 3000 | 3000 | 3000 | 3000 | 3000 |
| Pentanediol | | | | | |
| Niacinamide + | 3000 | 3000 | 3000 | 3000 | 1500 |
| Hexanediol | | | | | |
| Niacinamide + | 3000 | 3000 | 1500 | 3000 | 3000 |
| Heptanediol | | | | | |
| Niacinamide + | 1500 | 1500 | 1500 | 1500 | 750 |
| Caprylyl Glycol | | | | | |
| Niacinamide + | 1500 | 1500 | 1500 | 188 | 375 |
| Glyceryl Caprylate | | | | | |
| Niacinamide + | 600 | 300 | 300 | 150 | 150 |
| Decanediol | | | | | |

As can be seen or Table 2, the tested blends including C₃-C₁₀ diols as well as fatty acid glyceryl ester (glyceryl caprylate) show a significantly reduced MIC (about 10 fold reduction). From the tested blends caprylyl glycol, glyceryl caprylate and decanediol showed a superior antimicrobial activity in combination with niacinamide.

Based on these initial experiments, the inventors focused on compositions based on niacinamide with caprylyl glycol, glyceryl caprylate and decanediol and/or combinations thereof as potential synergistic preservatives.

In order to evaluate the potential preservative activity, the inventors performed numerous challenge tests for various combinations and controls (i.e. similar compositions without niacinamide).

Exemplary antimicrobial composition comprising Niacinamide and Glyceryl Caprylate at a w/w ratio of about 18.7:1 and further containing Decanediol at a w/w ratio of 1:3 relative to Glyceryl Caprylate (GC) has been tested in various cosmetic formulations including a non-ionic cream, cationic mask, anionic shampoo. When applied at a final w/w concentration of 3% the tested antimicrobial composition showed solid preservative activity in all the tested formulations (conforming with both USP and EP acceptance criteria). In contrast, same composition applied at a final w/w concentration of 1% didn't show a sufficient preservative activity. Moreover, the application of 5% of the tested antimicrobial composition resulted in an inferior activity, as compared to the application of only 3%. Further, sole application of Glyceryl Caprylate and Decanediol (without niacinamide) didn't show any markable preservative activity. Accordingly, it has been concluded that the preferable application range of the niacinamide/glycol-based antimicrobial compositions is about 3%, or between 2 and 5%.

Further, the inventors have tested the antimicrobial compositions with different niacinamide: GC ratios (namely 56:3, 29:3 and 129:3). At both ratios 56:3 and 29:3, the antimicrobial compositions showed solid antimicrobial activity in the tested formulation, wherein at a ratio of 56:3 the tested composition showed a superior preservative effect conforming with both USP and EP acceptance criteria. At 129:3 niacinamide: GC ratio the resulting composition showed inferior antimicrobial activity, especially with respect to contamination by molds (C. albicans and A. brasiliensis). Accordingly, it has been concluded that the preferable niacinamide: GC ratio in the antimicrobial composition is between about 10:1 and 30:1.

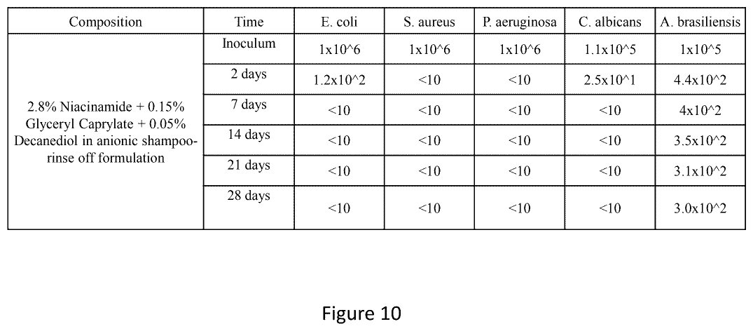

The results of a challenge test performed for antimicrobial composition with a niacinamide: GC ratio of 56:3, and further including decanediol are presented in FIG. 10.

Additionally, antimicrobial composition comprising Caprylyl Glycol (CG) as the diol (at niacinamide: CG ratio of 9:1) has been tested in various cosmetic formulations and showed solid preservative activity when applied at a final w/w concentration of 3%. In contrast, sole application of CG (without niacinamide) didn't show any markable preservative activity. Further, the inventors observed that the addition of Caprylhydroxamic acid (CA) at a w/w ratio of 1:3 relative to CG significantly improved the preservative activity of the resulting antimicrobial composition against A. brasiliensis.

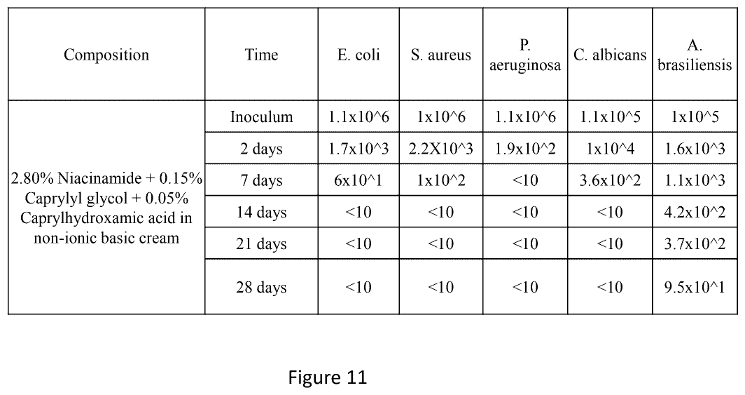

The results of a challenge test performed for antimicrobial composition including niacinamide, CG and CA are presented in FIG. 11.

To this end, a synergistic antimicrobial effect of niacinaminde with various glycols (e.g. GC, CG) with or without addition of CA or decanediol has been demonstrated.

### Example 3

The inventors have tested a potential preservative activity of a combination comprising niacinamide and polylysine at niacinamide: polylysine ratio of about 57:1. The results presented in Table 3 below demonstrate solid synergistic antimicrobial activity of the tested combinations (FICI score between 0.1 and 0.5 for the tested microorganisms). Of note, the MIC of niacinamide (as the sole representative) was between about 15000 and 30000 for all the tested microorganisms.

TABLE 3

| Microorganism | Combination MIC (ppm)/FICI score |
|---|---|
| E. coli | 468 |
| | FICI = 0.5 |
| S. aureus | 468 |
| | FICI = 0.5 |
| P. aeruginosa | 468 |
| | FICI = 0.5 |
| C. albicans | 468 |
| | FICI = 0.1 |
| A. brasiliensis | 468 |
| | FICI = 0.3 |

Further, the inventors have tested the antimicrobial compositions with different niacinamide: polylysine ratios (namely 84:1, 42:1 and 29.4:1) at the same final concentration of about 3% w/w in the non-ionic cream formulation. All the tested ratios showed solid preservative activity in the tested formulation. In contrast, even at the highest concentration of 0.1% (corresponding to polylysine concentration used in the 29.4:1 ratio-based formulation), polylysine as a standalone treatment didn't show any markable preservative activity.

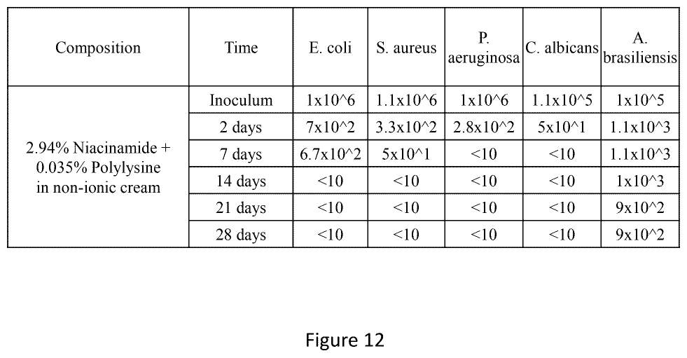

The results of a challenge test performed for antimicrobial composition including niacinamide and polylysine at a ratio of 84:1 are presented in FIG. 12.

Moreover, the inventors observed that the addition of 1:1 hexylresorcinol (relative to polylysine), significantly improves the preservative activity of the antimicrobial composition, especially against mold (A. brasiliensis).

To this end, based on the experimental results it was concluded that the exemplary antimicrobial compositions of the invention (Examples 1-3) showed a substantial synergistic effect, by substantially increasing antimicrobial activity, compared to single treatments with any one of the abovementioned constituents.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. (canceled)

2. (canceled)

3. (canceled)

4. (canceled)

5. (canceled)

6. (canceled)

7. (canceled)

8. (canceled)

9. (canceled)

10. An antimicrobial composition comprising niacinamide, a salt thereof or both; and a glycol; wherein:
a ratio between niacinamide, a salt thereof or both and said glycol is between 30:1 and 5: 1w/w by weight; and
wherein said antimicrobial composition is characterized by an antimicrobial synergistically effective amount of between about 2 and about 5% w/w.

11. The antimicrobial composition of claim 10, being a preservative composition characterized by a sterilizing or preservation activity within an article when present within said article at the antimicrobial synergistically effective amount.

12. The antimicrobial composition of claim 10, wherein said glycol comprises (i) C4-C15diol and (ii) R—C(O)—O—X or both, wherein R is an allyl between 3 and 20 carbon atoms long; X is a C2-C10 alkyl substituted by 2 hydroxy groups (ii).

13. The antimicrobial composition of claim 12, wherein said (ii) is a C5-C18 fatty acid monoglyceride.

14. The antimicrobial composition of claim 13, wherein said C5-C18 fatty acid monoglyceride is glyceryl caprylate.

15. The antimicrobial composition of claim 10, wherein the antimicrobial synergistically effective amount is between about 2.5 and 4% w/w; wherein said glycol comprises glyceryl caprylate and said C4-C15diol; wherein a w/w ratio between niacinamide and glyceryl caprylate is between about 8:1 and about 20:1; and wherein a w/w ratio between glyceryl caprylate and said C4-C15diol is between 1:1 and 5:1.

16. The antimicrobial composition of claim 11, wherein said diol glycol comprises said C4-C15diol; and wherein said C4-C15diol comprises a C4-C15 alkyl or heteroalkyl substituted by two hydroxy groups.

17. The antimicrobial composition of claim 16, further comprising Caprylhydroxamic acid and wherein a w/w ratio between said C4-C15diol and Caprylhydroxamic acid is between 1:1 and 5:1.

18. (canceled)

19. The antimicrobial composition of claim 11, wherein said C4-C15diol comprises any one of: caprylyl glycol, ethylhexyl glycerin and decanediol, or any combination thereof.

20. The antimicrobial composition of claim 10, wherein said antimicrobial composition is an aqueous composition; wherein a concentration of niacinamide and of said glycol with said aqueous composition is between 10 and 60% w/w and between 0.5 and 5% w/w, respectively; and wherein said aqueous composition further comprises a between 0.5 and 5% w/w of a surfactant; wherein the antimicrobial composition is characterized by a sterilizing or preservation activity within an article when applied to said article at the antimicrobial synergistically effective amount; and wherein said article is selected from a liquid formulation and a semi-solid formulation; and wherein the antimicrobial synergistically effective amount is sufficient for at least 20% increased reduction of a pathogen load within said article, as compared to sole addition of the same amount of niacinamide; wherein the antimicrobial synergistically effective amount is a preservative effectiveness according to EP and/or USP acceptance criteria, as determined by challenge test.

21. The antimicrobial composition of claim 12, wherein the C4-C15diol is decanediol and wherein R—C(O)—O—X is glyceryl caprylate; and wherein a w/w ratio between niacinamide and glyceryl caprylate is between between about 15:1 and about 20:1.

22. An antimicrobial composition comprising niacinamide, a salt thereof or both; and a polylysine; wherein:
a ratio between niacinamide, a salt thereof or both and said polylysine is between 100:1 and 20:1 by weight; and
wherein said antimicrobial composition is characterized by an antimicrobial synergistically effective amount of between about 2 and about 5% w/w.

23. The antimicrobial composition of claim 22, further comprising hexylresorcinol; optionally wherein a w/w ratio between said polylysine and hexylresorcinol is between 2:1 and 1:2.

24. (canceled)

25. (canceled)

26. The antimicrobial composition of claim 22, wherein said antimicrobial composition is an aqueous composition; wherein the ratio between niacinamide, a salt thereof or both and said polylysine is between 60:1 and 50:1 by weight; wherein said polylysine is characterized by an average MW between 2000 and 10000 Da.

27. The antimicrobial composition of claim 1, characterized by a sterilizing or preservation activity within an article when applied to said article at the antimicrobial synergistically effective amount; and wherein said article is selected from a liquid formulation and a semi-solid formulation; and wherein the antimicrobial synergistically effective amount is sufficient for at least 20% increased reduction of a pathogen load within said article, as compared to sole addition of the same amount of niacinamide; wherein the antimicrobial synergistically effective amount is a preservative effectiveness according to EP and/or USP acceptance criteria, as determined by challenge test.

28. (canceled)

29. (canceled)

30. (canceled)

31. (canceled)

32. (canceled)

33. (canceled)

34. (canceled)

35. (canceled)

36. A method for reducing pathogen load in and/or on an article or for preserving the article, the method comprising contacting said article with a synergistically effective amount of the antimicrobial composition of claim 1.

37. The method of claim 36, wherein said article is in a form of a moist textile substrate, liquid formulation or a semi-solid formulation, optionally wherein the article is a cosmetic product, a pharmaceutical product, a personal care product, or any combination thereof; and is characterized by a water content of at least 10%.

38. The method of any one of claim 36, wherein said reducing comprises at least 10 times CFU reduction of one or more pathogens within said article 48h after said contacting, compared to a similar article devoid of said antimicrobial composition; wherein said preserving is according to EP and/or USP acceptance criteria, as determined by a challenge test.

39. (canceled)

40. The method of claim 36, wherein said synergistically effective amount is sufficient for obtaining a dry weight concentration of the antimicrobial composition within said article of between about 2 and about 5% w/w; and wherein said synergistically effective amount is sufficient for obtaining a dry weight concentration of the antimicrobial composition within said article of between about 2.5 and about 4% w/w.

41. (canceled)
